(19) United States
(12) Patent Application Publication
(10) Pub. No.: US 2023/0374067 A1
(43) Pub. Date: Nov. 23, 2023

(54) NOVEL PEPTOIDS AND USE THEREOF FOR PREVENTING OR TREATING CHRONIC PAIN

(71) Applicants: Université Clermont Auvergne, CLERMONT-FERRAND (FR); Centre National de la Recherche Scientifique, PARIS (FR); SIGMA Clermont, AUBIÈRE (FR); Institut National de la Santé et de la Recherche Médicale, PARIS (FR); Université de Montpellier, MONTPELLIER (FR)

(72) Inventors: Jérôme BUSSEROLLES, Saulzet (FR); Emmanuel BOURINET, SAINT MATHIEU DE TREVIERS (FR); Claude TAILLEFUMIER, CHIDRAC (FR); Olivier ROY, COURNON D'AUVERGNE (FR); Lionel NAUTON, CHAMPEIX (FR); Youssef AISSOUNI, RIOM (FR)

(21) Appl. No.: 18/248,240

(22) PCT Filed: Sep. 30, 2021
(86) PCT No.: PCT/FR2021/051694

§ 371 Date: Apr. 6, 2023

(30) Foreign Application Priority Data
Oct. 6, 2020 (FR) 2010186

(51) Int. Cl. C07K 5/10; A61P 25/02

(52) U.S. Cl. CPC C07K 5/10; A61P 25/02

(57) ABSTRACT

The invention concerns new peptidomimetic (or peptoid) molecules for the prevention and/or treatment of chronic pain, particularly that resulting from peripheral neuropathies.

### CROSS-REFERENCE TO RELATED APPLICATIONS

The present application is a filing under 35 U.S.C. 371 as the National Stage of International Application No. PCT/FR2021/051694, filed Sep. 30, 2021, entitled “NOVEL PEPTOIDS AND USE THEREOF FOR PREVENTING OR TREATING CHRONIC PAIN,” which claims priority to French Application No. 2010186 filed with the Intellectual Property Office of France on Oct. 6, 2020, both of which are incorporated herein by reference in their entirety for all purposes.

### TECHNICAL FIELD OF THE INVENTION

The invention relates to novel peptidomimetic molecules (or peptoids, also called peptide-peptoid hybrids or peptomers) for the prevention and/or treatment of chronic pain, in particular that resulting from peripheral neuropathies (e.g. induced by chemotherapy).

In the description below, the references in square brackets ([ ]) refer to the list of references at the end of the text.

### STATE OF THE ART

Chronic pain affects 1.5 billion people worldwide for whom the therapeutic response is clearly insufficient (Finnerup NB et al., 2015) [1]. Recent epidemiological studies have estimated that chronic pain affects approximately 20% of adults and 50% of older adults (von Hehn et al., 2012) [2]. Globally, 60% of people with pain are less able to work and 20% report having lost their jobs due to pain. At the same time, 20 billion units of analgesic drugs were sold in 2010, up from 14 billion in 2005 (+9.3%). In 2017, the global pain market reached $61.05 billion and is expected to be worth $77.13 billion by 2023, growing at an annual growth rate of 4% from 2017 to 2023.

Neuropathic pain affects about 7% of the general population and 25% of patients with chronic pain. There are two types of neuropathic pain: peripheral neuropathic pain (depending on its origin, it may involve the territories of a plexus, a root, or a trunk, or be more diffuse in the context of polyneuropathy) and central neuropathic pain (lesions affecting the sensory pathways or pain control). Peripheral neuropathy induced by chemotherapy, which is a frequent adverse effect of many anticancer agents, is characterized by significant chronic pain that has a lasting effect on the patient's quality of life and leads to dosage adjustments with the risk of reduced clinical efficacy. The analgesic pharmacopoeia, which is becoming poorer (withdrawal of Di-antalvic in 2011), is characterized by old and often badly tolerated treatments.

Innovation has been stalled for more than 50 years, with the majority of new drugs proposed being simple reformulations or combinations of existing drugs. Recent meta-analyses show that no treatment for neuropathic pain is really effective to date with the exception of gabapentin and duloxetine with a still low efficacy score (Hershman et al., 2014) [3].

The family of HCN ion channels (Ludwig et al., Nature 1998; Santoro et al., 1998; Seifert et al., 1999) [4-6], which includes four HCN1-4 members, may offer excellent opportunities for the development of new heart rate-lowering drugs and novel analgesics. HCN channels are widely distributed in pain pathways and play an important role in the development and maintenance of neuropathic pain (Dunlop et al., 2009; Lewis et al., 2011) [7, 8]. Non-selective HCN blockers have been shown to decrease pain symptoms in rodent models of neuropathic pain (Descoeur et al., 2011; Young et al., 2014) [9, 10]. Their cardiac and visual side effects (e.g. pan-HCN blockers), however, limit the clinical translation of their use to treat neuropathic pain.

Interestingly, HCN channel function is tightly regulated by an auxiliary subunit, the Rab8b interaction protein (TRIP8b) that is not expressed in the heart. This cytoplasmic protein binds to the C-terminus of HCN channel subunits via two contact sites (Lewis et al., 2009) [11]. Furthermore, the atomic structure of the tetratricopeptide (TPR) region of TRIP8b in complex with a 6 amino acid peptide representing the C-terminus of HCN2 was determined by X-ray crystallography (Bankston et al., 2012) [12].

In the current context, where the majority of work is focused on developing inhibitors acting directly on HCN channels, targeting TRIP8b and/or the TRIP8b-HCN interaction could offer a new possibility to modulate HCN activity in pain states without the risk of affecting cardiac and visual functions.

### DESCRIPTION OF THE INVENTION

The present invention is based on the hypothesis that the disruption of TRIP8b-HCN interactions leading to a decrease in currents (Ih) induced by HCN channels, could have an analgesic effect in rodent models of acute and chronic peripheral neuropathies induced by oxaliplatin, faithfully reproducing human symptomatology.

The proof of efficacy of this strategy was initially validated using NUCC-5953, a molecule from a 20,000-molecule library screen, capable of disrupting the TRIP8b-HCN interaction (Han et al., 2015) [13]. The Inventors thus demonstrated that oxaliplatin-induced acute cold hypersensitivity is reversed in a dose-dependent manner by intrathecal (FIG. 2) or systemic (FIG. 3) injection of NUCC-5953. No alteration of locomotor activity was observed. Preliminary data also showed that administration of NUCC-5953 reduced membrane addressing, and thus the activity of HCN1 and 2 observed in neuropathic animals (FIG. 5B). Furthermore, in vitro application of NUCC-5953 showed no effect on HCN activation-induced currents in cardiac cells (in contrast to nonspecific blockers of HCN channels, FIG. 6).

The inventors then decided to design specific blockers of the TRIP8b-HCN interaction based on the interaction mode of the two proteins. However, while protein-protein interactions (PPIs) play an essential role at all levels of cellular function, making them undeniably therapeutic targets, the design of potent PPI inhibitors remains a real challenge. Low molecular weight molecules, although widely screened, are poorly suited to compete with interactions involving large surfaces (>800 Å). Furthermore, to design effective inhibitors, it is important to be able to mimic the protein/protein recognition segment in its bioactive «hot segment» conformation. Peptides are the compounds of choice to design these new inhibitors provided that they can solve the problems of metabolic and conformational stability, but also of cellular penetration to reach intracellular targets.

The inventors then turned to peptidomimetic chemistry via the synthesis of peptoids (N-substituted glycine oligomers) which are particularly adapted to the development of inhibitors targeting a protein-protein interaction, and which can also be called peptomers (i.e. oligomers combining “peptoid” monomers and “amino-acid” monomers—Ostergaard et al 1997 [17]—i.e. peptide-peptoid hybrids).

These are synthetic peptide mimetics whose oligomeric nature allows for great modularity in terms of size and chemical diversity.

They allow a fine structural design and have a better bioavailability than peptides (protease resistance, better membrane permeability, non-immunogenic skeleton).

The Inventors have therefore designed new peptoids targeting the interaction between the TPR domains of TRIP8b and the C-terminus of HCN, capable of exerting an analgesic effect in a model of oxaliplatin-induced acute neuropathy without adverse cardiac or visual effects.

The present invention therefore is directed to a peptoid of the following general formula (I):

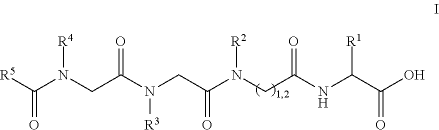

where
R¹ is independently CH₃ (L-Ala), CH(CH₃)₂ (L-Val), CH(CH₃)CH₂CH₃ (L-Ile), CH₂CH(CH₃)₂ (L-Leu and D-Leu), CH₂C(CH₃)₃, C(CH₃)₃, CH₂(cyclobutyl);
R², R³, and R⁴ are the side chains of the peptoid units incorporated sequentially during the synthesis from primary amines;
R² and R³ are independently selected from: CH₂CH(CH₃)₂, CH₂CH₂NH₂, CH₂CH₂OH, CH₂CH₂CH₂OH, CH₂CH(OH)CH₃, CH₂CONH₂, CH₂CH₂CONH₂. The side chains with an alcohol or amine function have been incorporated into the oligomers in protected form: silyl ether for the alcohol function and tert-butyl carbamate (Boc) for the amine function;
R⁴ is an aliphatic or aromatic group selected from:

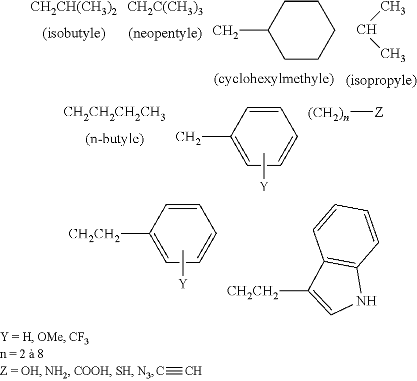

R⁵ is independently CH₃, CH(CH₃)₂, CH₂C₆H₅, C₆H₄X (where X in the ortho, meta or para position is independently H, OMe, CF₃, CH₃, CH₂CH₃, F, Br, Cl, NO₂), 3-indolyl, 3-quinolinyl.

Advantageously, R⁴ is an aliphatic or aromatic group selected from:

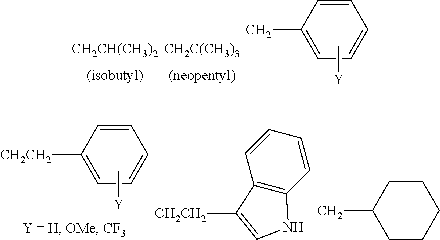

Advantageously, R⁵ is independently CH₃, CH₂C₆H₅, C₆H₄X (where X in ortho, meta or para position is independently H, OMe, CF₃, CH₃, CH₂CH₃, F, Br, Cl), 3-indolyl, 3-quinolinyl.

Advantageously, the peptoids of the present invention are selected from peptoids of general formula (I) wherein:
R¹ is independently CH₃ (L-Ala), CH(CH₃)₂ (L-Val), CH(CH₃)CH₂CH₃ (L-Ile), CH₂CH(CH₃)₂ (L-Leu and D-Leu), CH₂C(CH₃)₃, C(CH₃)₃, CH₂(cyclobutyl);
R², R³, and R⁴ are the side chains of the peptoid units incorporated sequentially during the synthesis from primary amines;
R² and R³ are independently selected from: CH₂CH₂NH₂, CH₂CH₂OH, CH₂CH₂CH₂OH, CH₂CH(OH)CH₃, CH₂CONH₂, CH₂CH₂CONH₂. The side chains with an alcohol or amine function have been incorporated into the oligomers in protected form: silyl ether for the alcohol function and tert-butyl carbamate (Boc) for the amine function;
R⁴ is an aliphatic or aromatic group selected from:

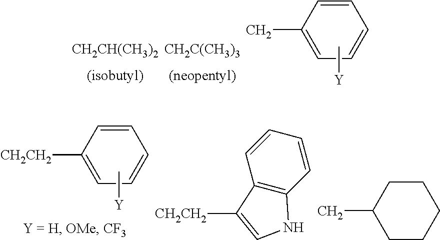

R⁵ is independently CH₃, CH₂C₆H₅, C₆H₄X (wherein X in ortho, meta or para position is independently H, OMe, CF₃, CH₃, CH₂CH₃, F, Br, Cl), 3-indolyl, 3-quinolinyl.

Preferably, the peptoids of the present invention, of general formula (I), have 2 carbon atoms on the second unit (starting from the C-terminus). Advantageously, the peptoids of the present invention are selected from the peptoids of the following general formula (I′):

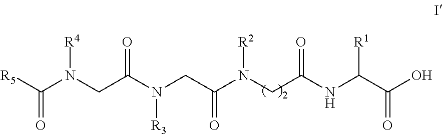

where
R¹, R², R³, R⁴ and R⁵ are as defined above.

According to a particular embodiment, the peptoids of the present invention have the following formulae:

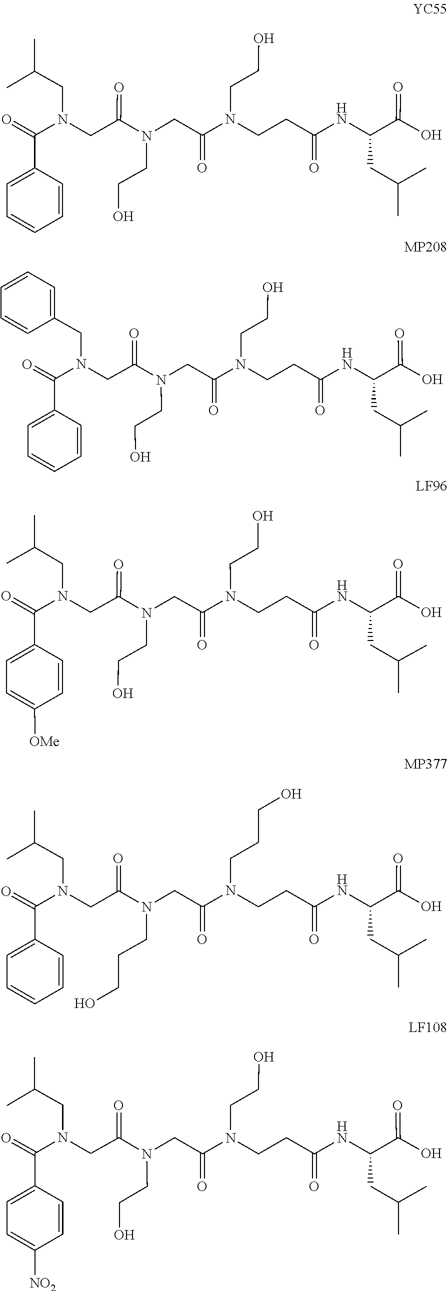

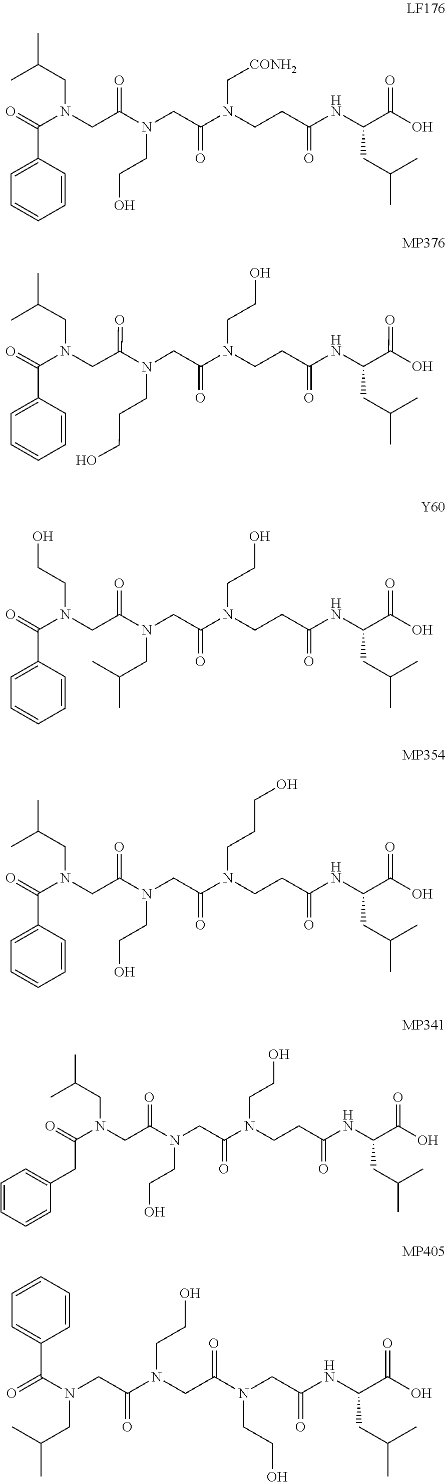

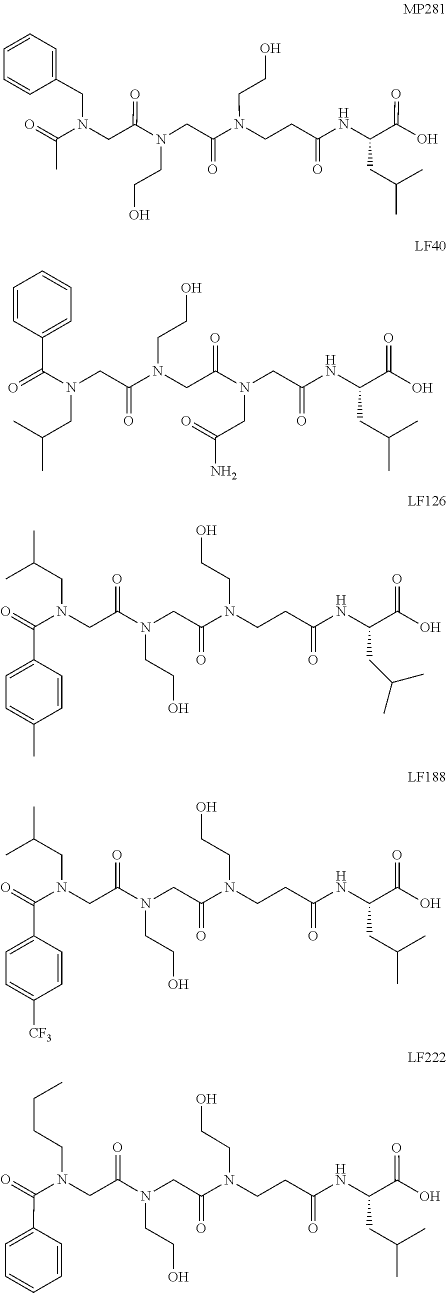

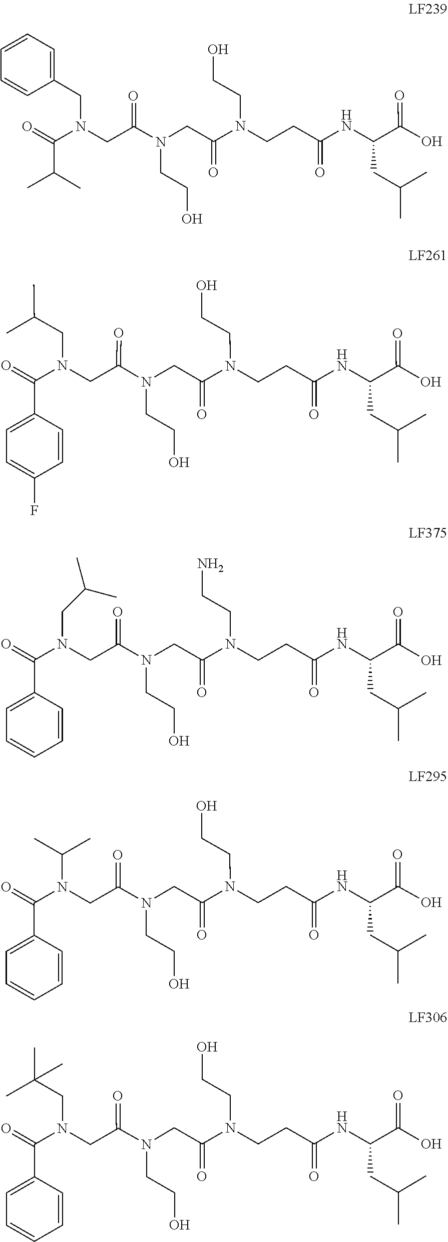 [SMILES: CC(C)C[C@H](NC(=O)CCN(CCO)C(=O)CN(CCO)C(=O)CN(Cc1ccccc1)C(=O)C(C)C)C(=O)O ; CC(C)C[C@H](NC(=O)CCN(CCO)C(=O)CN(CCO)C(=O)CN(CC(C)C)C(=O)c1ccc(F)cc1)C(=O)O ; CC(C)C[C@H](NC(=O)CCN(CCN)C(=O)CN(CCO)C(=O)CN(CC(C)C)C(=O)c1ccccc1)C(=O)O ; CC(C)C[C@H](NC(=O)CCN(CCO)C(=O)CN(CCO)C(=O)CN(C(=O)c1ccccc1)C(C)C)C(=O)O ; CC(C)C[C@H](NC(=O)CCN(CCO)C(=O)CN(CCO)C(=O)CN(CC(C)(C)C)C(=O)c1ccccc1)C(=O)O]

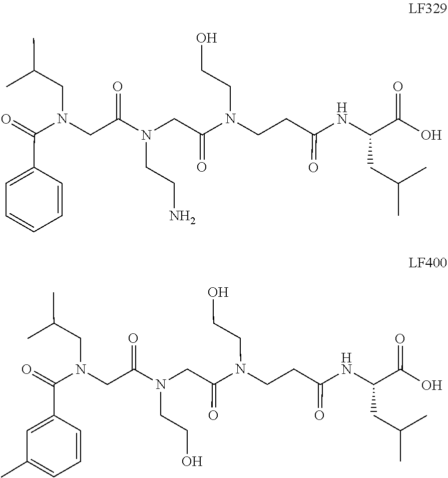

The present invention also relates to an intermediate for the synthesis of the peptoid of general formula (I), said peptoid comprising 3 units. Said synthesis intermediate is a peptoid of the following general formula (II):

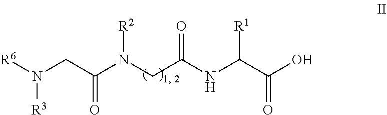

où
R¹, R² and R³ are as defined above ;
R6 is H, R³, COR⁵ (where R⁵ is as defined above) or R⁷;
R⁷ is independently a group among:

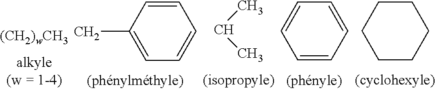

In particular, the present invention is directed to peptoids of formula (II) wherein R⁶ is H.

In particular, the present invention is directed to peptoids of formula (II) in which R⁶ is COR⁵.

In particular, the present invention is directed to peptoids of formula (II) wherein R⁶ is R⁷.

In particular, the present invention is directed to peptoids of formula (II) in which R⁶ is R³.

Advantageously, the peptoids according to the invention may be selected from the peptoids of the following general formulae (IIa), (IIb), (IIc) and (IId):

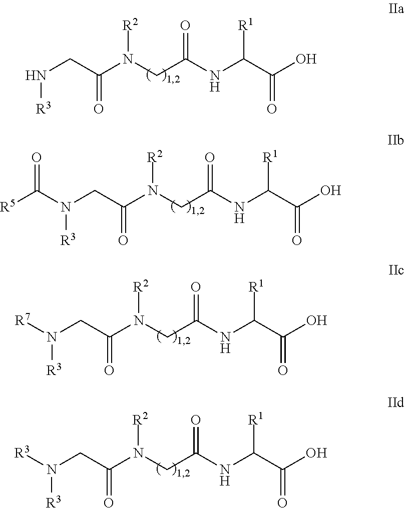

wherein
R¹, R², R³, R⁵ and R⁷ are as defined above.

Preferably, the peptoids of the present invention of formula (II), (IIa), (IIb), (IIc) or (IId) have 2 carbon atoms on the second unit (starting from the C-terminus). Advantageously, the peptoids are selected from the peptoids of the following formulae (II′), (II′a), (II′b), (II′c) or (II′d):

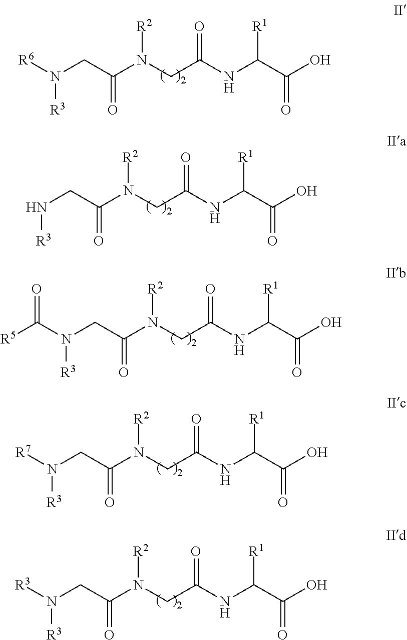

wherein
R¹, R², R³, R⁵, R⁶ and R⁷ are as defined above.

The present invention is also directed to a pharmaceutical composition comprising a peptoid according to the present invention, and a pharmaceutically acceptable carrier.

The present invention is also directed to a peptoid or a pharmaceutical composition according to the present invention for use as a medicament.

The present invention is also directed to a peptoid or a pharmaceutical composition according to the present invention for use in the prevention or treatment of chronic pain, in particular chronic pain of neuropathic origin, more particularly peripheral neuropathic pain for example induced by chemotherapy.

In a context where therapeutic innovation in the field of analgesics is at a standstill, the peptoids of the present invention and their therapeutic potential may have a significant impact on improving the quality of life of patients suffering from neuropathic pain induced by anticancer drugs and possibly their survival. Furthermore, as HCN channels are also involved in the etiology of various neuropathic and inflammatory pain conditions, the peptoids of the present invention pave the way for new classes of analgesic drugs that are more broadly active on various types of chronic pain.

### BRIEF DESCRIPTION OF THE FIGURES

FIGS. 1A-E shows the expression of HCN1 (A), HCN2 (B) and TRIP8b (C) proteins in DRG neurons 4 days after vehicle or oxaliplatin (6 mg/kg, i.p.) administration. TRIP8b protein expression in the dorsal horn of the spinal cord 4 days after vehicle or oxaliplatin (6 mg/kg, i.p.) administration (D) or after repeated (twice weekly for 3 weeks) vehicle or oxaliplatin administration (E) N=3/group, Mann-Whitney test.

FIGS. 2A-C represents (A) study design (B) time course of mean +/−SEM of paw immersion latency threshold (in sec) and (C) area under the curve (AUC, sec. min) of the dose-dependent analgesic effect in OIPN (oxaliplatin-induced neuropathy model mice) (n=8) treated with vehicle or NUCC5953 (0.2, 1 or 5 μg, i.t.); *p<0.05, **p<0.01, ***p<0.001, vs. veh. group, Two-way ANOVA and Tukey post hoc test.

FIGS. 3A-C represents (A) study design (B) time course of mean +/−SEM of paw immersion latency (sec) and (C) area under the curve (AUC, sec. min) of the dose-dependent analgesic effect in OIPN mice (n=8) treated with vehicle, NUCC5953 (4 mg/kg, s.c.) or duloxetine (30 mg/kg, s.c.); *p<0.05, **p<0.01, ***p<0.001, vs. veh. group, Two-way ANOVA and Tukey post hoc test.

FIGS. 4A-B represents histograms showing the latency to fall (A) and to fall speed (B) of mice treated with vehicle or NUCC5953 (4 mg/kg, s.c.) subjected to a rotarod test with increasing speed (from 4 to 40 rpm for 10 min).

FIGS. 5A-B represents (A) study design (B) subcellular (membrane) expression of HCN1, HCN2, and TRIP8b in DRGs from animals treated with vehicle (V), oxaliplatin (O), or oxaliplatin+NUCC5953 (5 nmol, i.t.) (N).

FIG. 6 represents the lack of effect of NUCC5953 on If current amplitude in contrast to ivabradine (VIA).

FIG. 7 represents the heart rate of mice (HR): (A) baseline value, (B) average heart rate for 30 minutes after injection of NUCC5953 (4 mg/kg, s.c.), and (C) average value at the end of 30 minutes; values are mean +/−SEM (n=3).

FIG. 8 represents the molecular dynamics of TRIP8b interacting with the SNL peptide.

FIGS. 9A-C represents (A) study design (B) time course of mean +/−SEM of paw immersion latency (sec) and (C) area under the curve (AUC, sec. min) of the dose-dependent analgesic effect in OIPN mice (n=8) treated with vehicle or peptoid YC55 (0.2, 1 or 5 μg, i.t.); *p<0.05, **p<0.01, ***p<0.001, vs. veh. group, Two-way ANOVA and Tukey post hoc test.

FIGS. 10A-C represents (A) study design (B) time course of mean +/−SEM of paw immersion latency threshold (in sec) and (C) area under the curve (AUC, sec. min) of the dose-dependent analgesic effect in OIPN mice (n=8) treated with vehicle or peptoid YC55 (2.5, 5 or 10 mg/kg, s.c.); *p<0.05, **p<0.01, ***p<0.001, vs. veh. group, Two-way ANOVA and Tukey post hoc test.

FIGS. 11A-B represents histograms showing the latency to fall (A) and to fall speed (B) of mice treated with vehicle, YC55 (2.5, 5, or 10 mg/kg, s.c.), or valium (2.5 mg/kg, s.c.) subjected to a rotarod test at increasing speed (4 to 40 rpm for 10 min). ***p<0.001 T30 min post administration (▪) vs T0 (□) for a given molecule, Student's t-test.

FIGS. 12A-C represents (A) study design (B) Time course of mean +/−SEM of mechanical thresholds (in g) and paw immersion latency threshold (in sec) and (C) area under the curve (AUC, sec. min) of PTX rats (n=7-9) treated with vehicle or peptoid YC55 (0.2, 1 or 5 μg, i.t.); *p<0.05, **p<0.01, ***p<0.001, vs. veh group, Two-way ANOVA and Tukey post hoc test.

FIGS. 13A-B represents (A) study design (B) Time course of mean +/−SEM of paw immersion latency threshold (in sec) of neuropathic rats (n=7-9) treated with vehicle (◯) or peptoid YC55 (▪) (5 μg, i.t.); ***p<0.001, vs. veh. group, two-way ANOVA and Tukey's post hoc test.

FIGS. 14A-C represents (A) study design (B) Time course of mean +/−SEM of paw immersion latency threshold (in sec) and (C) area under the curve (AUC, sec. min) of the analgesic effect of OIPN mice (n=7-9) treated with vehicle or peptoids MP208, MP405, or YC55 (5 μg, i.t.); *p<0.05, **p<0.01, ***p<0.001, vs. veh. group, Two-way ANOVA and Tukey post hoc test.

FIGS. 15A-C depicts (A) study design (B) Time course of mean +/−SEM of paw immersion latency threshold (sec) and (C) area under the curve (AUC, sec. min) of the analgesic effect of OIPN mice (n=7-9) treated with vehicle or peptoids MP376, MP354, or MP341 (10 mg/kg, s.c.); *p<0.05, **p<0.01, ***p<0.001, vs. veh. group, Two-way ANOVA and Tukey post hoc test.

FIGS. 16A-C represents (A) study design (B) Time course of mean +/−SEM of paw immersion latency threshold (in sec) and (C) area under the curve (AUC, sec. min) of the analgesic effect of OIPN mice (n=7-9) treated with vehicle or peptoids LF108, LF306, or LF188 (5 μg, i.t.); *p<0.05, **p<0.01, ***p<0.001, vs. veh. group, Two-way ANOVA and Tukey post hoc test.

FIGS. 17A-C represents (A) study design (B) Time course of mean +/−SEM of paw immersion latency threshold (in sec) and (C) area under the curve (AUC, sec. min) of the analgesic effect of OIPN mice (n=7-9) treated with vehicle or peptoids LF295, LF400, or LF329 (5 μg, i.t.); *p<0.05, **p<0.01, ***p<0.001, vs. veh. group, Two-way ANOVA and Tukey post hoc test.

FIGS. 18A-C represents (A) study design (B) Time course of mean +/−SEM of paw immersion latency threshold (in sec) and (C) area under the curve (AUC, sec. min) of the analgesic effect of OIPN mice (n=7-9) treated with vehicle or peptoids LF126, LF261, or LF275 (5 μg, i.t.); *p<0.05, **p<0.01, ***p<0.001, vs. veh. group, Two-way ANOVA and Tukey post hoc test.

FIGS. 19A-C represents (A) study design (B) Time course of mean +/−SEM of paw immersion latency threshold (in sec) and (C) area under the curve (AUC, sec. min) of the analgesic effect of OIPN mice (n=7-9) treated with vehicle or peptoids LF222, LF239, or LF176 (5 μg, i.t.); *p<0.05, **p<0.01, ***p<0.001, vs. veh. group, Two-way ANOVA and Tukey post hoc test.

FIGS. 20A-C represents (A) study design (B) Time course of mean +/−SEM of paw immersion latency threshold (sec) and (C) area under the curve (AUC, sec.min) of analgesic effect of OIPN mice (n=7-9) treated with vehicle or peptoid LF369 (5 μg, i.t.); * p<0.05, **p<0.01, ***p<0.001, vs. veh. group, Two-way ANOVA and Tukey's post hoc test.

### EXAMPLES

### Example 1

### Material and Methods

### Animals

Experiments were performed on 20-24 g male C57BI/6JRj mice or 150-175 g male Sprague-Dawley rats provided by Janvier Laboratories (France), maintained on a 12 h light/dark cycle and fed and watered ad libitum. Behavioral experiments were performed blindly, in a quiet room, by the same experimenter for a given test, taking great care to minimize or avoid animal discomfort. All animal procedures were approved by the local animal ethics committee and experiments were performed in accordance with the guidelines provided by the European Community on the care and use of animals (Directive 2010/63/EU).

### Behavioral Tests

Von Frey test: Mechanical sensitivity was assessed with calibrated Von Frey filaments of 0.07 g, 0.6 g or 1.4 g. The filaments were applied five times in order of increasing stiffness, perpendicularly to the plantar surface of the hind paw and pressed until they bent. The number of responses for a given filament force was counted.

Immersion test: The tail or paw was immersed in a water bath set at 10° C. or 46° C. until shrinkage was observed (cut-off time: 30 s). The average of two separate determinations of withdrawal latency was calculated (Janssen, Niemegeers & Dony, 1963) [14].

Paw pressure test in rats: Rats were subjected to the paw pressure test previously described by Randall and Selitto (1957) [15]. Nociceptive thresholds, expressed in grams, were measured with a Ugo Basile analgesic meter (Apelex, probe tip diameter 1 mm, weight 30 g) by applying increasing pressure to the right hind paw of the rats until a pain sign (vocalization threshold) was obtained (cut-off was 750 g). Before the treatments, the rats were habituated to the test by handling them without subjecting them to paw pressure. Then, after obtaining two consecutive stable vocalization threshold values, the treatment effects were evaluated after 15, 30, 45, 60, 90, and 120 min. Results are expressed as vocalization thresholds, in grams. To investigate the overall effects, the areas under the time course curves (AUC, g.min) of the antihyperalgesic effects were calculated from the individual scores at each time point, using the trapezoidal method. Data were analyzed by a 2-way ANOVA followed by a Bonferroni test, when the time course of the effects was studied. A one-way ANOVA followed by a Student Newman-Keuls test was used to analyze the effect of the different treatments determined by the CSAs. The level of statistical significance was set at p<0.05.

Rotarod Test: For the Rotarod test (Bioseb), the animals were first habituated to remain on the stationary cylinder for 10 min before switching to another 10 min with a rotational speed of 4 rpm. The animals were given the drug immediately after training and the test was performed 30 min later. The test consisted of measuring the latency to fall of the animals as the rotation increased from 4 to 40 rpm linearly over 5 min. The average of the two closest values over 3 trials was calculated.

In vivo heart rate recording: ECG telemetry recordings were performed using TA10EA-F20 telemetry transmitters (DSI) in a subcutaneous pouch with paired wire electrodes placed on the thorax as previously published (Mesirca et al., 2014) [27].

### Animal Pain Model

Oxaliplatin-induced acute neuropathy in mice: Mice were injected intraperitoneally (i.p.) with 6 mg/kg oxaliplatin dissolved in 5% glucose directly after the first behavioral assessment. At 90 h after drug administration, corresponding to the time required to reach peak hyperalgesia (Descoeur et al., 2011) [9], a second assessment of cold sensitivity and mechanical sensitivity was performed.

Acute paclitaxel-induced neuropathy in rats: Rats were injected i.p. with dimethyl sulfoxide (DMSO; solvent) or 1 mg/kg/1 mL paclitaxel (Sigma-Aldrich, Lyon, France) on days D0, D2, D4, and D7. The behavioral test was performed on the day of the first paclitaxel injection (D0) and on days 10 (D10) and 14 (D14) after paclitaxel injection.

Spinal nerve ligation induced neuropathy in rats: According to the procedure of Kim and Chung (1992) [16], rats were placed in the supine position and anesthetized with 10 mg/kg xylazine and 75 mg/kg ketamine (i.p). The fur on the left side of the spine was then shaved to expose the skin. The skin was cut parallel to the spine and the paravertebral muscles were separated. The L5 transverse apophysis was removed until the L5 spinal nerve was exposed. The L5 nerve was isolated and ligated with silk thread. Finally, the muscle and skin incisions were sutured and the wound was disinfected with iodophor and 75% ethyl alcohol. In the sham group, the rats underwent the same surgical procedure without the nerve ligation.

### Ex Vivo Tests

RNA isolation and quantification: DRG pairs from lumbar segments L4 to L6 were dissected and pooled. RNA was extracted using TRIzol® reagent (Nitrogen) according to the manufacturer's instructions (Thermofisher). RNA quality was assessed by the Agilent 2100 Bioanalyzer (Agilent Technologies), samples with RIN (RNA Integrity Number) greater than 8 were retained for further analysis. Reverse transcription was performed with SuperScript II reverse transcriptase according to the protocol provided by Invitrogen. For real-time quantitative PCR, amplification was performed using the Light Cycler Fast Start DNA kit (Roche diagnostics) and the amount of DNA in real-time was measured with a Mastercycler Realplex thermal cycler (Eppendorf). The relative amount of cDNA in each triplicate was averaged using a standard concentration curve and normalized to the GUS gene cDNA.

Amplification conditions were as follows: preincubation for 7 min at 95° C. followed by 50 cycles of 20 s at 95° C. and 20 s at the specific hybridization temperature of the primer pair followed by 20 s at 72° C.

RNA sequencing: We used RNA sequencing analysis on L4-L5-L6 DRGs from mice treated with oxaliplatin (3 mg/kg, i.p.) or vehicle twice a week for 3 weeks. At the end of the experiment (D21), mice were anesthetized and killed, and L4-L5-L6 DRGs were rapidly harvested, frozen in liquid nitrogen and stored at −80° C. until use. Total RNAs from L4-L5-L6 DRGs were extracted using the RNeasy Micro kit (Qiagen) according to the manufacturer's protocol and Ribosomal RNA removed using Ribo-Zero Plus rRNA depletion kit (Illumina). RNA concentration was determined using the Take3 Nucleic Acid Quantification Plate (Epoch, BioTek, USA). RNA integrity was analyzed by a capillary electrophoresis instrument (Fragment Analyser, Agilent®) and sent to Fasteris (https://www.fasteris.com) for RNA sequencing. Libraries were prepared using the Illumina TruSeq protocol and SBS-based sequencing was performed using a HiSeq 2500 platform (Illumina). The analysis was performed in different steps: splice junction mapping (TopHat2), counting (HTSeq-count), filtering, normalization (edgeR, DESeq and DESeq2) and differential analysis (edgeR, DESeq and DESeq2) performed by Benjamin Bertin and Yoan Renaud (GreD, Clermont-Ferrand).

Membrane Fractionation:Tissues were homogenized in HEPES buffer (20 mM HEPES pH 7.4, 320 mM sucrose, 5 mM EDTA, 5 mM EGTA) containing protease inhibitors at 4° C., sonicated and centrifuged at 1000 g for 15 min at 4° C. The pellets were discarded. For organelle spin, supernatants were centrifuged at 15,000 g for 10 min at 4° C. and organelle pellets were retained. For cytosolic spin, supernatants were centrifuged at 100,000 g for 1 h at 4° C. The cytosolic fraction was in the supernatants and the membrane fraction in the pellets. The membrane pellets were then resuspended in SB buffer (20 mM HEPES pH 7.4, 1M Kl, 5 mM EDTA, 5 mM EGTA at 4 degrees) and centrifuged at 100,000 g for 1 hour at 4° C. to remove weakly bound membrane proteins. The membrane fractions were then resuspended in RB buffer (20 mM HEPES pH 7.4, 100 mM NaCl, 5 mM EDTA, 5 mM EGTA) containing Triton (1%) and protease inhibitors. After sonication in an ice bath, the membrane fractions were subsequently subjected to gentle agitation for 2 hours at 4° C. The solubilized membrane fractions were centrifuged at 100,000 g for 1 hour at 4° C. The membrane fraction was in the supernatant.

Western blot: L4 to L6 DRGs from three mice were pooled and homogenized in lysis buffer containing triton (1%) and protease inhibitors. Homogenates were centrifuged at 18,000 g 20 min at 4° C. and supernatants collected. 50 μg of total protein was separated by SDS-PAGE on a 7.5% gel. After transfer to nitrocellulose membrane and saturation with BSA (5%), primary antibodies were used at a dilution of 1/500 for HCN1 and HCN2 (Neuromab), 1/1000 for TRIP8b (Neuromab), and 1/5000 for Actin□ (Sigma-Aldrich). After 3 successive washes with TBST, the corresponding HRP-coupled secondary antibodies (ThermoFisher) were incubated at a 1:10,000 dilution and detected with the chemiluminescent substrate (SuperSignal West Pico Chemiluminescent Substrate, Thermofisher).

Patch-clamp recording: The electrophysiology experiments were performed on isolated pacemaker heart cells as previously described (Marger et al., 2011) [28]. The whole-cell patch-clamp technique was used to record If currents generated by HCN channels, employing an Axopatch 200A amplifier (Axon Instruments Inc., Foster USA).

Recording electrodes of ˜5 m MΩ resistance were filled with an intracellular solution containing (mM/L): K+-aspartate, 130; NaCl, 10; ATP-Na+ salt, 2; creatine phosphate, 6.6; GTP-Mg2+, 0.1; CaCl2, 0.04 (pCa=7); Hepes-KOH, 10; (pH=7.2 with KOH). A Tyrode extracellular solution containing 5 mM BaCl2 to block IK1 currents was used. The effect of NUCC5953 (5 μM) on If current amplitude and kinetics was compared with that of ivabradine (3 μM), both diluted in the extracellular solution. Data acquisition was performed using pClamp software (ver. 9, Axon Instruments Inc.).

### In Vitro Assays

Fluorescence polarization assay: a fluorescence polarization (FP) based assay was used to identify compounds capable of disrupting the interaction between HCN and the TRIP8b tail, as published (Han et al., 2015) [13].

### Example 2

### Results

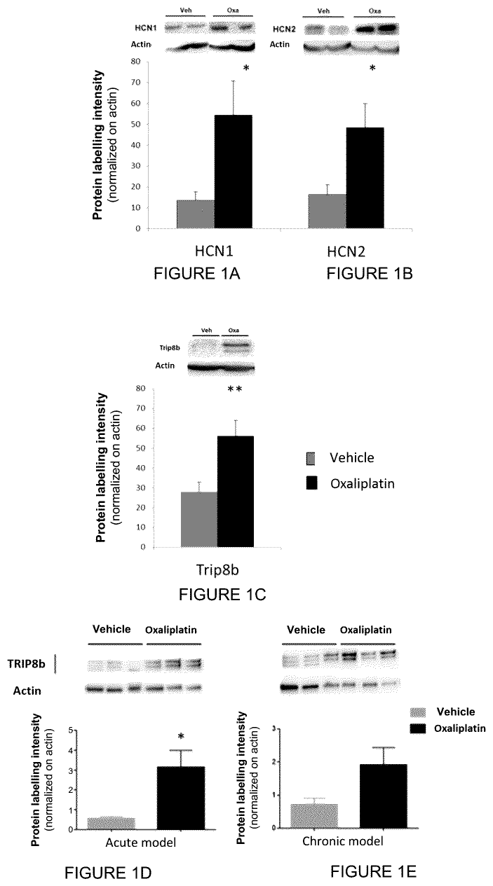

For the first time the presence of TRIP8b mRNA (RNAseq data obtained from DRG mice showed the presence (FPKM>1) of splice variants 2-5: NM_01163516, NM_01163517, NM_01289505, NM_01310460) and protein in DRG and TG neurons in mice was observed. In addition, transcript and protein expression of HCN1-2 channels and TRIP8b were significantly increased in peripheral neurons of oxaliplatin-treated animals compared with controls, suggesting the involvement of the 2 proteins in the development of OIPN (oxaliplatin-induced neuropathy model mouse) (FIGS. 1A-C). Analysis of TRIP8b protein expression in the spinal cord of animals treated with a single (FIG. 1D) or after repeated administration (FIG. 1E) of oxaliplatin versus vehicle shows a clear increase in TRIP8b protein under the effect of oxaliplatin.

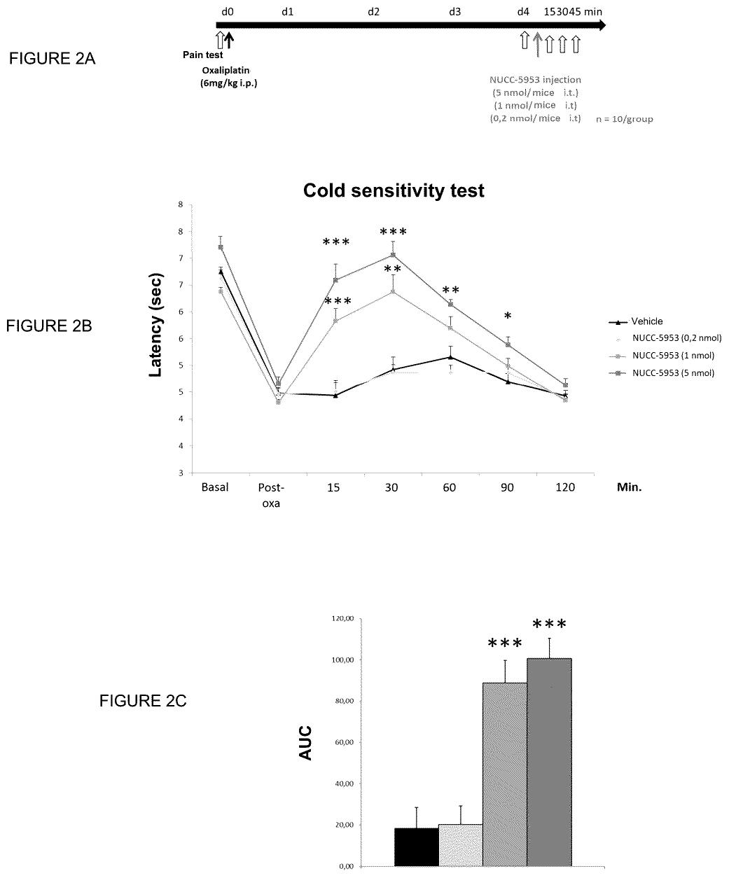

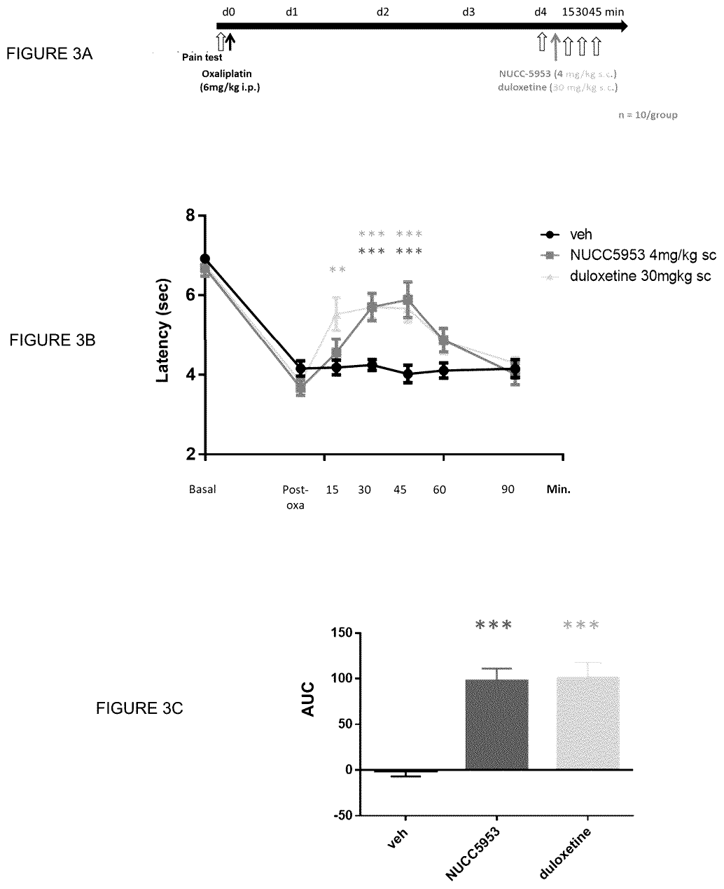

Using NUCC5953, a molecule capable of disrupting the TRIP8b-HCN interaction (Han et al., 2015) [13], it was shown that oxaliplatin-induced acute cold hypersensitivity can be reversed in a dose-dependent manner either i.t. (0.2; 1; 5 μg/mouse; FIG. 2) or systemically (4 mg/kg; FIG. 3).

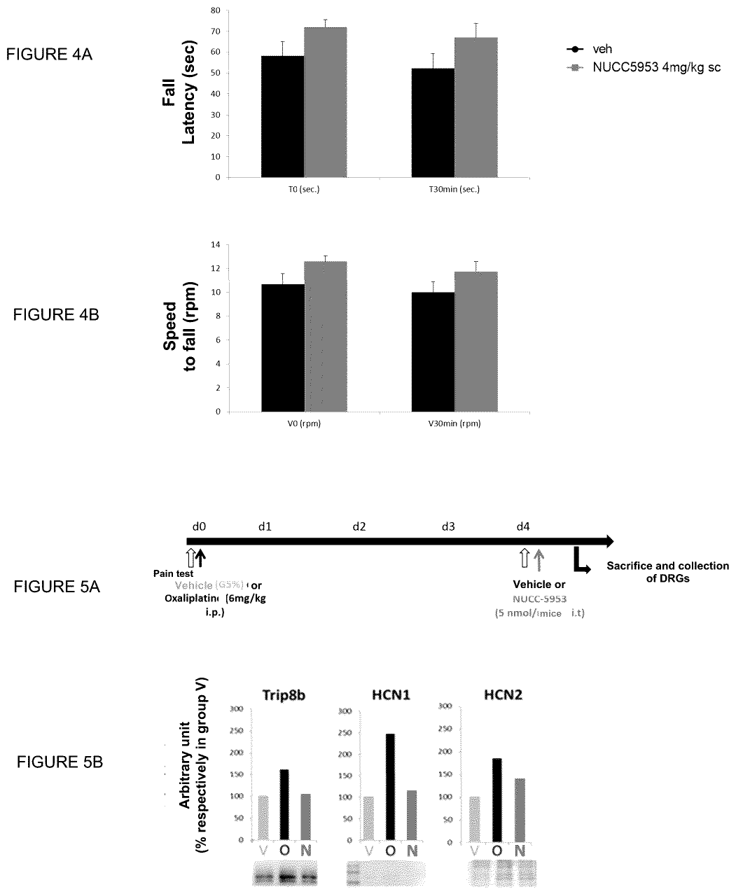

No alteration in locomotor activity was observed in the Rotarod test when mice were treated with NUCC5953 (4 mg/kg) administered subcutaneously (FIG. 4).

At the same time, preliminary data also suggest that administration of NUC5953 reduces the overexpression of HCN1 and 2 and TRIP8b proteins in the membrane fraction of DRG neurons from OIPN animals (FIG. 5).

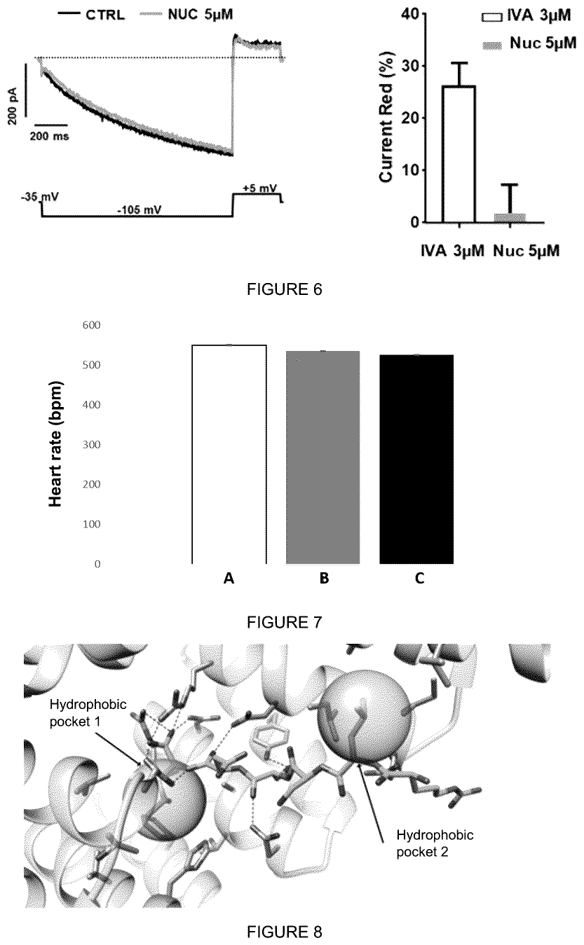

Patch-clamp experiments on isolated cardiac pacemaker cells showed no effect of NUCC5953 on If current amplitude, in contrast to the pan-HCN blocker Ivabradine (FIG. 6).

NUCC5953 does not affect heart rate in vivo when administered at an analgesic dose (4 mg/kg, i.p.) (FIG. 7). The results show that the average heart rate is not altered in three mice before (A), at the time of NUCC5953 injection (B) and 30 minutes after injection (C).

### Example 3

### Design of Peptidomimetics

Design of peptidomimetics

A—Tetrameric Peptidomimetics

Peptidomimetic compounds mimicking the sequence of the carboxy-terminus of the HCN2 channel were designed and synthesized. The design of these peptidomimetics was based on an extensive X-ray structure study of a co-crystal of TRIP8b with the C-terminal region of HCN2, available in the protein database (pdb: 4EQF). Preparatory modeling work to complete and improve this structure was performed. The missing parts were modeled by sequence homology from different PEX5 structures (PDB: 1FCH, 3R9A, 4KXK, 4KYO, 2J9Q, 2COM and 2COL) showing sequence identity ranging from 30 to 60% with TRIP8b. The resulting model identified the contacts between residues responsible for the formation of the HCN-TRIP8b complex. In particular, this study identified two hydrophobic pockets in the TPR domain of TRIP8b, which are essential for good affinity of ligands to this protein domain (FIG. 8).

The hydrophobic pocket 1 of TRIP8b provides side-chain anchoring of the C-terminal residue of the ligands. Effective interaction of the ligands with hydrophobic pocket 2, located near the central region of TRIP8b, identified as a binding site for the CNBD domain of HCN, also enhances the affinity of the inhibitors. Several 4-residue peptide-peptoid hybrid oligomers bearing various side chains capable of targeting these hydrophobic pockets were designed and evaluated by molecular dynamics (MD).

The structure-based design identified peptidomimetic ligands interacting at the SNL peptide binding site (TPR domains) of TRIP8b. These ligands have a formula that is represented by the above LF108 formula or the following general formula (I):

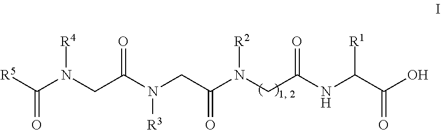

where
R¹, R², R³, R⁴ and R⁵ are as defined above.

The compounds are peptide-peptoid hybrid oligomers (peptomers) (Ostergaard et al., 1997) [17]. They comprise four residues including an α-amino acid positioned at the C-terminus of the sequence, and three consecutive peptoid residues. The third residue (from the N-terminus) is either a β-peptoid unit or an α-peptoid unit. The α-peptoids generally referred to as “peptoids” refer to N-substituted glycine oligomers (Simon et al., 1992) [18]. Compared to peptides, the side chains are attached to the nitrogen atoms of the amide bonds rather than to the α-carbon atoms. The β-peptoids are oligomers composed of N-substituted β-aminopropionic acid units (Hamper et al., 1998) [19]. Thus, a β-peptoid residue contains an additional methylene group in the backbone compared to an α-peptoid residue. Peptoids have advantageous characteristics for the development of bioactive compounds. They are amenable to solid-phase synthesis, using an iterative “submonomer” approach in which each new unit is constructed in two steps (Zuckermann et al., 1992) [20]. Peptoids can incorporate a wide variety of side chains, both proteinogenic and non-natural side chains (Cuff et al., 2010) [21]. “Sub-monomer” syntheses use simple starting molecules such as bromoacetic acid and primary amines for the construction of α-units and acryloyl chloride and primary amines for the construction of β-peptoid units. The primary amines, from which the side chains are incorporated, are commercially available by the hundreds, or can be prepared prior to their use where appropriate. The peptoid backbone based on N,N-disubstituted amides (tertiary amides) has been shown to be resistant to proteases (Miller et al., 1995) [22]. The presence of tertiary amide linkages also gives them better cell permeability (Kwon et al., 2007; Vollrath et al., 2013) [23, 24], and a few studies tend to show that peptoids are less immunogenic than peptides (Li et al., 2010; Chongsiriwatana et al., 2008) [25, 26].

The syntheses were performed on 2-chlorotrityl chloride (2-CTC) resin leading to a carboxylic acid at the C-terminus of the peptomer after cleavage. The first residue immobilized on the support, an α-amino acid, was selected primarily from the group of hydrophobic proteinogenic amino acids, including L-leucine which corresponds to the C-terminus of the SNL peptide. The α-amino acids were immobilized on the resin in a protected N-Fmoc form. After removal of the Fmoc group, chain extension by peptoid residues was achieved by “submonomer chemistry” approaches. Known protocols were implemented but some technical adjustments were required for the synthesis of the β-peptoid monomer. The N-terminus was acylated prior to cleavage under acidic conditions with hexafluoroisopropanol (HFIP) and deprotection of side chains with trifluoroacetic acid (TFA) in dichloromethane.

The crude peptomeric compounds were analyzed by LC-MS and then purified on C18 flash chromatography cartridges with UV detection at 214 and 220 nm. The pure fractions were collected and lyophilized. Pure compounds were analyzed by reverse phase HPLC on Nucleodur® C4 column (5 μm, 300 Å, 250 mm×4.6 mm). Table 2 below shows the HRMS and HPLC data of the compounds.

TABLE 2

| | | | retention time | |
|---|---|---|---|---|
| | | | (min) | |
| | | | chromatograms | HPLC |
| Molecule | expected | | LCMSª | purity |
| reference | mass | observed mass/formula | or HPLC^{b} | (%)^{c} |
| YC55 | 564.3159 | 565.3227 [M + H]⁺/ | 22.02^{b} | 97 |
| | | C₂₈H₄₅N₄O₈ | | |
| YC60 | 564.3159 | 565.3229 [M + H]⁺/ | 3.74ª | n.d. |
| | | C₂₈H₄₅N₄O₈ | | |
| MP208 | 598.3003 | 599.3075 [M + H]⁺/ | 22.86^{b} | 92 |
| | | C₃₁H₄₃N₄O₈ | | |
| MP281 | 536.2846 | 537.2919 [M + H]⁺/ | 20.69^{b} | 96 |
| | | C₂₆H₄₁N₄O₈ | | |
| MP341 | 578.3316 | 579.3388 [M + H]⁺/ | 4.05ª | n.d. |
| | | C₂₉H₄₇N₄O₈ | | |
| MP354 | 578.3316 | 579.3388 [M + H]⁺/ | 3.81ª | n.d. |
| | | C₂₉H₄₇N₄O₈ | | |
| MP376 | 578.3316 | 579.3388 [M + H]⁺/ | 3.76ª | n.d. |
| | | C₂₉H₄₇N₄O₈ | | |
| MP377 | 592.3472 | 593.3545 [M + H]⁺/ | 3.81ª | n.d. |
| | | C₃₀H₄₉N₄O₈ | | |
| MP405 | 550.3003 | 551.3075 [M + H]⁺/ | 21.59^{b} | 85 |
| | | C₂₇H₄₃N₄O₈ | | |
| LF40 | 563.2955 | 564.3021 [M + H]⁺/ | 21.66^{b} | 91 |
| | | C₂₇H₄₂N₅O₈ | | |
| LF96 | 594.3265 | 595.3337 [M + H]⁺/ | 22.06^{b} | 92 |
| | | C₂₉H₄₇N₄O₈ | | |
| LF108 | 609.3010 | 608.2939 [M + H]⁻/ | 22.07^{b} | 92 |
| | | C₂₈H₄₂N₅O₁₀ | | |
| LF176 | 577.3112 | 578.1388 [M + H]⁺/ | 21.53^{b} | 97 |
| | | C₂₈H₄₄N₅O₈ | | |
| LF126 | 578.3316 | 579.3381 [M + H]⁺/ | 24.53^{b} | 89 |
| | | C₂₉H₄₇N₄O₈ | | |
| LF188 | 632.3033 | 633.3099 [M + H]⁺/ | 23.30^{b} | 98 |
| | | C₂₉H₄₄F₃N₄O₈ | | |
| LF222 | 564.3159 | 565.3229 [M + H]⁺/ | 21.55^{b} | 94 |
| | | C₂₈H₄₅N₄O₈ | | |
| LF239 | 564.3159 | 565.3228 [M + H]⁺/ | 21.12^{b} | 95 |
| | | C₂₈H₄₅N₄O₈ | | |
| LF261 | 582.3065 | 583.3138 [M + H]⁺/ | 21.34^{b} | 98 |
| | | C₂₈H₄₄FN₄O₈ | | |
| LF275 | 563.3319 | 564.3395 [M + H]⁺/ | 30.79^{b} | 94 |
| | | C₂₈H₄₆N₅O₇ | | |
| LF295 | 550.3003 | 551.3068 [M + H]⁺/ | 20.36^{b} | 86 |
| | | C₂₇H₄₃N₄O₈ | | |
| LF306 | 578.3316 | 579.3387 [M + H]⁺/ | 21.32^{b} | 95 |
| | | C₂₉H₄₇N₄O₈ | | |
| LF329 | 563.3319 | 564.3383 [M + H]⁺/ | 20.90^{b} | 95 |
| | | C₂₈H₄₆N₅O₇ | | |
| LF400 | 578.3316 | 579.3381 [M + H]⁺/ | 21.78^{b} | 91 |
| | | C₂₉H₄₇N₄O₈ | | |
| LF369 | 389.4490 | 390.2231 [M + H]⁺ | 18.32^{b} | 90 |
| | | C₁₇H₃₂N₃O₇ | | |

^{a}LCMS. UHPLC Ultimate 3000 RSLC chain (ThermoScientific), Kinetex EVO C18 column (100 × 2.1 mm; 1.7 μm) (Phenomenex), flow rate: 0.45 mL/min, column oven: 30° C., H₂O gradient (0.1% formic acid/ACN (0.1% formic acid): t = 0 min 95:5; t = 7.5 min 1:99, t = 8.5 min 1:99; t = 9 min 95:5; t = 11 min 95:5.
^{b}HPLC. Series 1100 Agilent chain, Nucleodur ® C4 column (5 μm, 300 Å, 250 mm × 4.6 mm), flow rate 0.5 mL/min. H2O/ACN gradient (0.1% TFA): t = 0 min 95:5; t = 5 min 95:5; t = 25 min 5:95; t = 35 min 5:95, t = 40 min 95:5, t = 50 min 95:5.
^{c}UV 214 nm detection.

### Side Chains

R¹ is independently CH₃ (L-Ala), CH(CH₃)₂ (L-Val), CH(CH₃)CH₂CH₃ (L-Ile), CH₂CH(CH₃)₂ (L-Leu and D-Leu), CH₂C(CH₃)₃, C(CH₃)₃, CH₂(cyclobutyl);
R², R³, and R⁴ are the side chains of the peptoid units incorporated sequentially during the synthesis from primary amines;
R² and R³ are independently selected from: CH₂CH(CH₃)₂, CH₂CH₂NH₂, CH₂CH₂OH, CH₂CH₂CH₂OH, CH₂CH(OH)CH₃, CH₂CONH₂, CH₂CH₂CONH₂. The side chains with an alcohol or amine function have been incorporated into the oligomers in protected form: silyl ether for the alcohol function and tert-butyl carbamate (Boc) for the amine function;
R⁴ is an aliphatic or aromatic group chosen from:

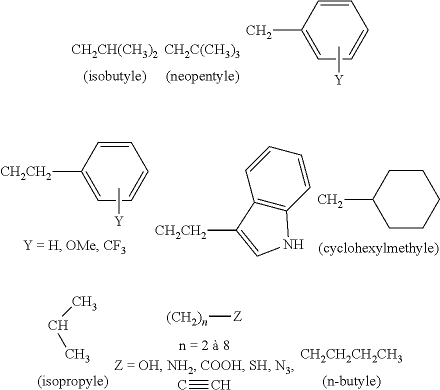

R⁵ is independently CH3, CH(CH3)2, CH2C6H5, C6H4X (where X in the ortho, meta or para position is independently H, OMe, CF3, CH3, CH2CH3, F, Br, Cl, NO2), 3-indolyl, 3-quinolinyl.
Representative Synthetic Scheme on 2-chlorotrityl Chloride Resin for the Synthesis of N-acylated Peptomers Incorporating an α-amino acid at the C-Terminus, Followed by a β- and two α-peptoid Residues.

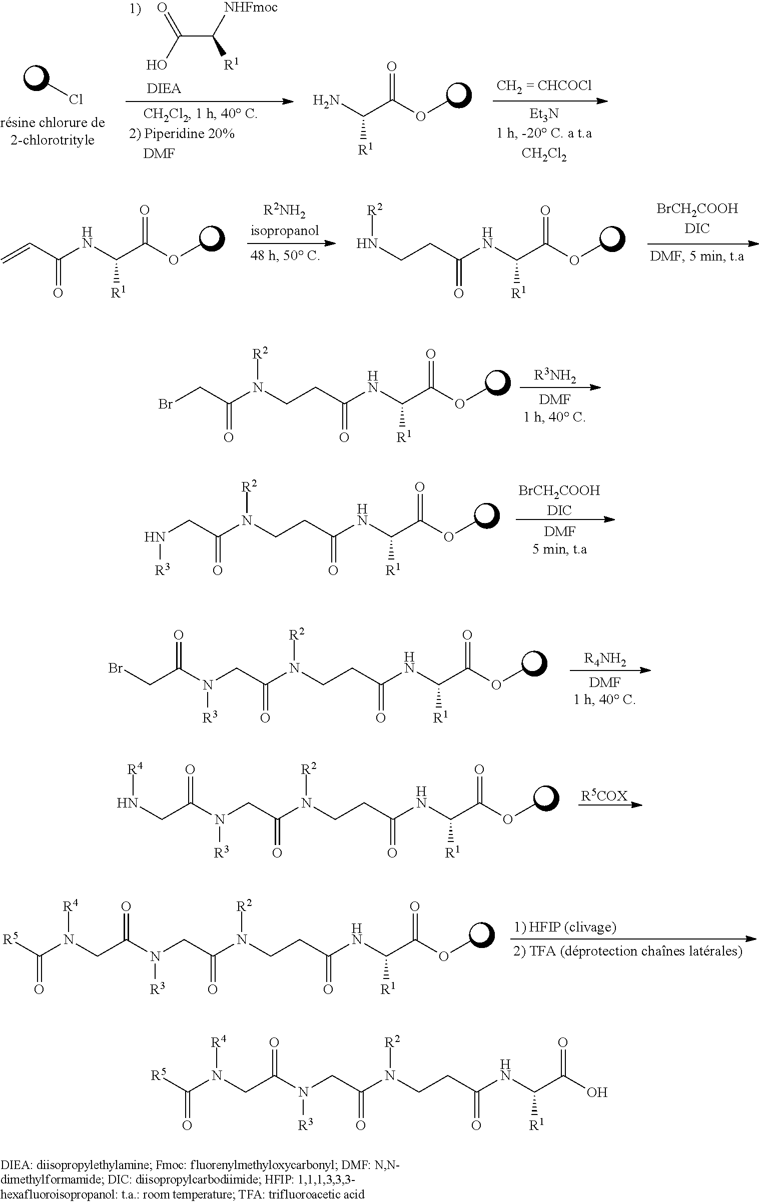

Synthesized molecules

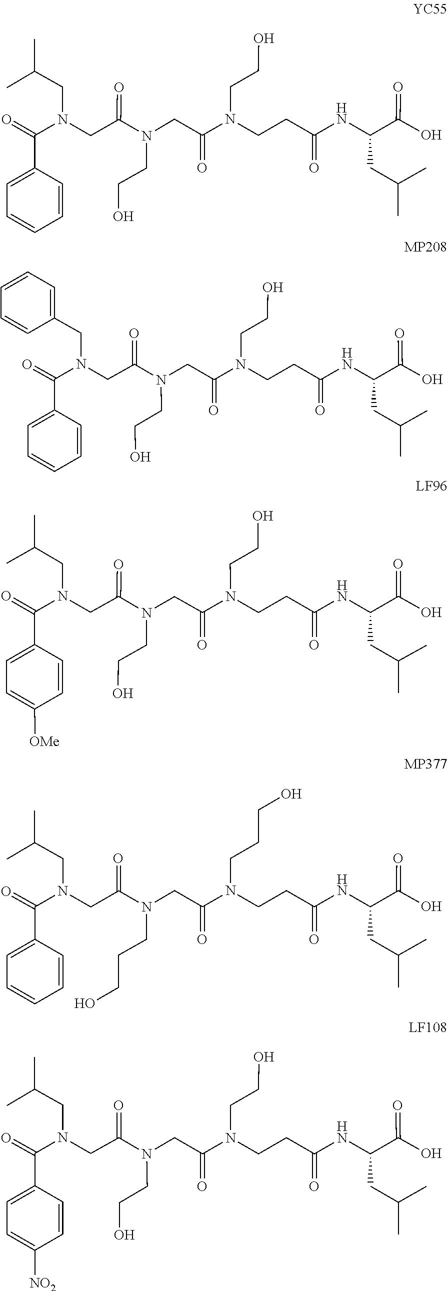

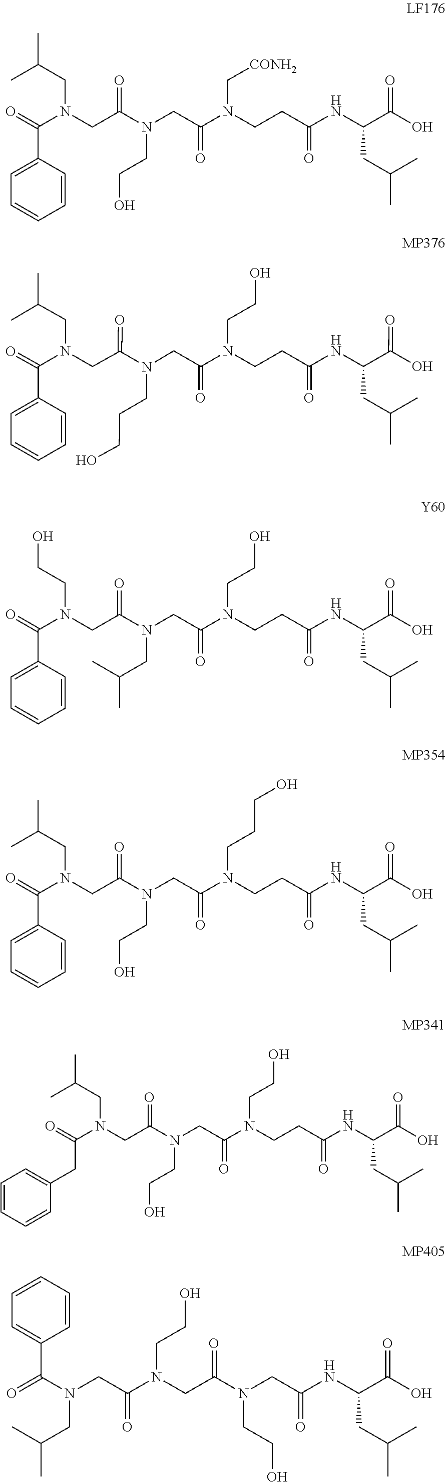

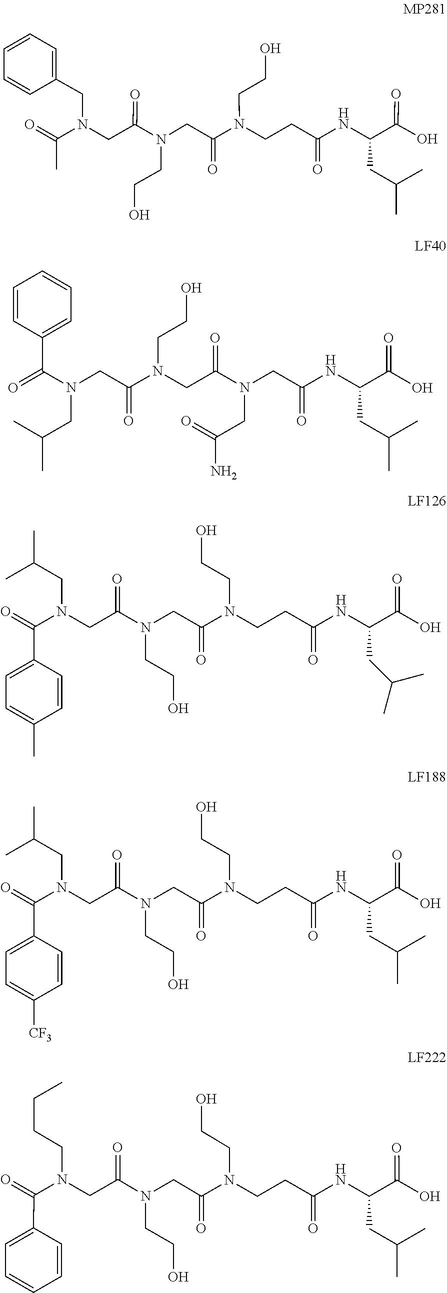

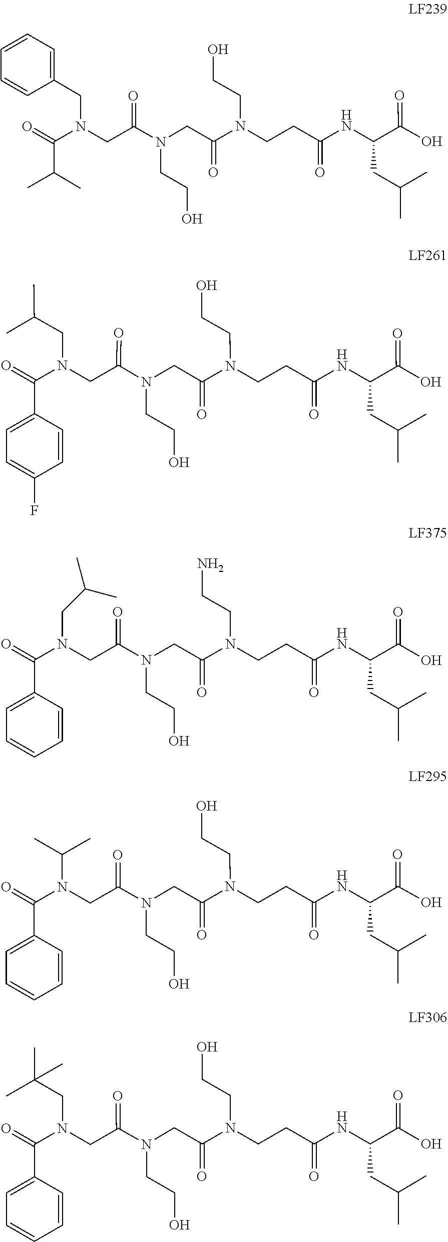 [SMILES: CC(C)C[C@H](NC(=O)CCN(CCO)C(=O)CN(CCO)C(=O)CN(Cc1ccccc1)C(=O)C(C)C)C(=O)O ; CC(C)C[C@H](NC(=O)CCN(CCO)C(=O)CN(CCO)C(=O)CN(CC(C)C)C(=O)c1ccc(F)cc1)C(=O)O ; CC(C)C[C@H](NC(=O)CCN(CCN)C(=O)CN(CCO)C(=O)CN(CC(C)C)C(=O)c1ccccc1)C(=O)O ; CC(C)C[C@H](NC(=O)CCN(CCO)C(=O)CN(CCO)C(=O)CN(C(=O)c1ccccc1)C(C)C)C(=O)O ; CC(C)C[C@H](NC(=O)CCN(CCO)C(=O)CN(CCO)C(=O)CN(CC(C)(C)C)C(=O)c1ccccc1)C(=O)O]

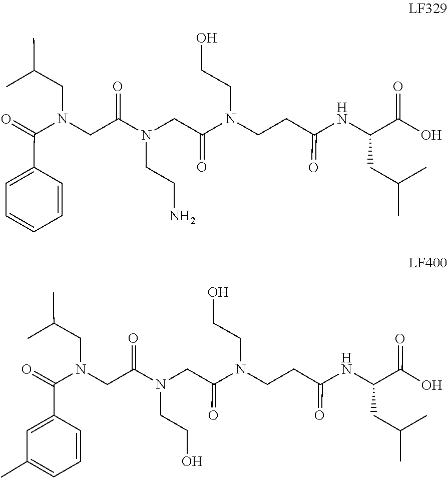

### Synthesis

Compounds corresponding to the general formula (I) were synthesized on 2-chlorotrityl chloride resin (Novabiochem 100-200 mesh) in 5 mL plastic syringes equipped with a sinter and stopcock. The syringes were placed on an orbital shaker heating platform during the reactions.

In a typical synthesis described with L-leucine at the C-terminus and a β-peptoid residue preceding this amino acid, 2-chlorotrityl chloride resin (1.33 mmol g−1, 0.100 g, 0.133 mmol) was swollen in 2 mL of dry CH₂Cl₂ (2×20 min). After filtration, 0.12 mL of a 2.2 M solution of Fmoc-Leu-OH in CH₂Cl₂ (2.0 equivalents) was added, followed by 2.0 mL of a 0.4 M solution of DIEA in CH₂Cl₂ (6.0 equivalents). The mixture was stirred for 40 min at 25° C. The resin was drained, washed with CH₂Cl₂ (2×2 mL), and the binding of Fmoc-Leucine to the resin was repeated under the same conditions. The resin was washed with CH₂Cl₂ (5×2 mL). The Fmoc group was removed by adding 2.0 mL of a 20% piperidine solution in DMF and stirring for 15 min at room temperature. The operation was repeated under the same conditions, after which the resin was washed with DMF (5×2 mL) and drained.

Introduction of the β-peptoid residue. The resin was cooled by two washes with CH₂Cl₂ at −20° C. 1.21 mL of a 0.22 M solution of acryloyl chloride (2.0 equivalents) in CH₂Cl₂ cooled to −20° C. was added, followed by 0.8 mL of a 0.5 M solution of Et3N (3.0 equivalents) in cooled CH₂Cl₂ (−20° C.). The resin was stirred for 1 h at 25° C., drained, and then treated again with acryloyl chloride under the same conditions. The resin was filtered and washed with CH₂Cl₂ (5×2 mL) and drained. The resulting acrylamide was treated with a 2.0 M solution of the amine R²NH₂ in isopropanol (12.0 equivalents) for 24 h at 50° C. After filtration of the resin, the aza-Michael reaction was repeated under the same conditions. The resin was washed with isopropanol (5×2 mL) before submonomer synthesis of two α-peptoid residues.

Synthesis of α-peptoid residues. To the resin were added 2.0 mL of a 0.4 M solution of bromoacetic acid in DMF (6.0 equivalents), followed by 2.13 mL of a 0.5 M solution of CID in DMF (8.0 equivalents). After stirring for 5 min at 25° C., the resin was drained and washed with DMF (5×2 mL). The resulting bromoacetamide was then treated with a 2.0 M solution of the amine R³NH₂ (20.0 equiv) in DMF and the mixture was left on the stirring platform for 1 h at 40° C. The resin was filtered and washed with DMF (5×2 mL). The last two bromoacetylation and substitution steps were repeated under the same conditions to construct the fourth residue bearing the desired R⁴ side chain. The N-terminus of the peptomer was then acylated according to one of the general procedures described below, prior to its removal from the resin.

For cleavage, the resin was treated with 1.5 mL of a 2.4 M solution of HFIP in CH₂Cl₂. After stirring for 40 min at 25° C., the resin was filtered and washed with CH₂Cl₂ (5×2 mL). The solvent was removed under reduced pressure. Finally, the silylated or Boc protecting groups of the side chains were removed by treating the peptomer with 2 mL of 25% (v/v) TFA in CH₂Cl₂ for 10 min at room temperature. The reaction mixture containing the final product was diluted with CH₂Cl₂ (5 mL) before complete evaporation in vacuo. In order to remove all traces of TFA, the product dissolved in CH2Cl2 was evaporated under vacuum, repeating this operation 5 times.

### General Procedures for Acylation of the N-Terminus of the Resin-Bound Peptomer

Acetylation: To the resin was added 1.56 mL of a 1.7 M solution of acetic anhydride in DMF (20.0 equivalents), followed by 0.8 mL of a 2.0 M solution of DIEA in DMF (12.0 equivalents). After stirring for 90 min at 25° C., the resin was filtered and washed with DMF (5×2 mL).

Benzoylation: To the resin was added 0.8 mL of a 1.0 M solution of benzoyl chloride in CH₂Cl₂ (6.0 equiv.), followed by 0.8 mL of a 2.0 M solution of DIEA in CH₂Cl₂ (12.0 equiv.,). After stirring for 40 min at 25° C., the resin was drained and washed with CH₂Cl₂ (5×2 mL).

Acylation from carboxylic acids: In a typical coupling reaction described here from phenylacetic acid, 2.0 mL of a 0.4 M solution of phenylacetic acid in DMF (6.0 equiv.) and 2.13 mL of a 0.5 M solution of DIC (8.0 equiv.) in DMF were added successively to the resin. After stirring for 1 h at 25° C., the resin was drained and washed with DMF (5×2 mL).

Of the compounds (YC55, YC-60) that were synthesized, YC55 demonstrated equal potency to NUCC5953 in disrupting the TRIP8b-HCN interaction (Table 1: IC50 of peptoids).

TABLE 1

| | YC55 | YC60 | Acox3 |
|---|---|---|---|
| IC50 | 8.862 | 12.71 | 0.6029 |

The analgesic effect of novel peptidomimetic compounds injected either intrathecally or systemically has been demonstrated in oxaliplatin-induced acute neuropathic pain in mice.

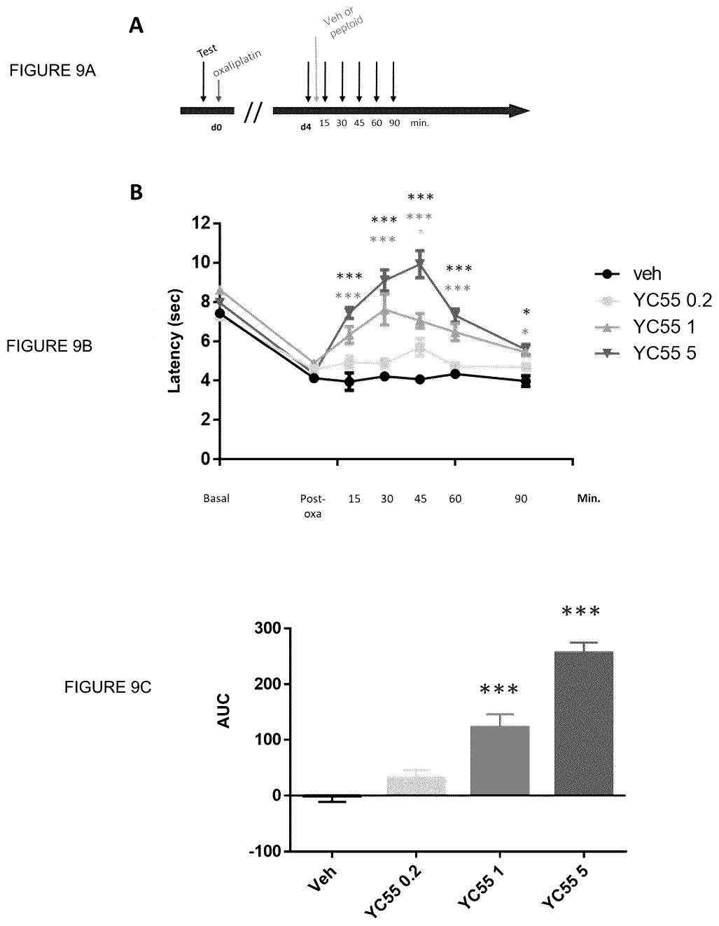

Thus, the lead compound YC55 demonstrated a dose-dependent analgesic effect in OIPN mice when administered i.t. (FIG. 9).

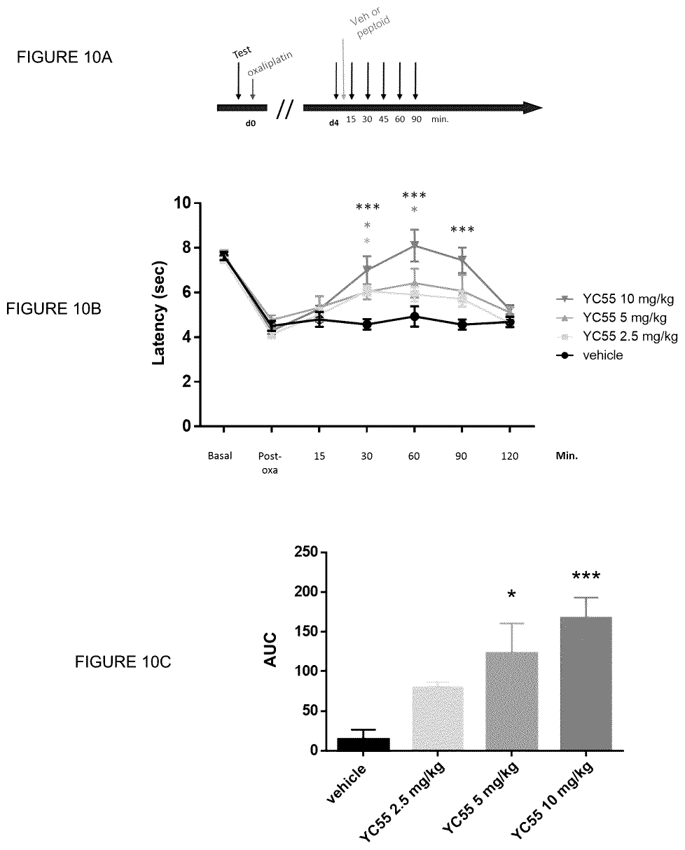

Compound YC55 also demonstrated a dose-dependent analgesic effect in OIPN mice when administered s.c. (FIG. 10).

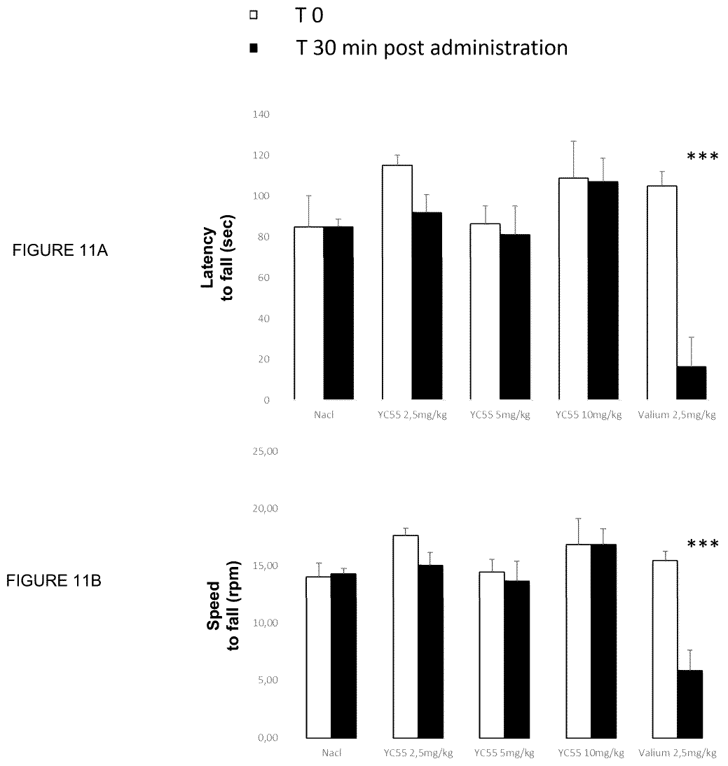

No alteration in locomotor activity was observed in the Rotarod test when mice are treated with YC55 administered subcutaneously (FIG. 11).

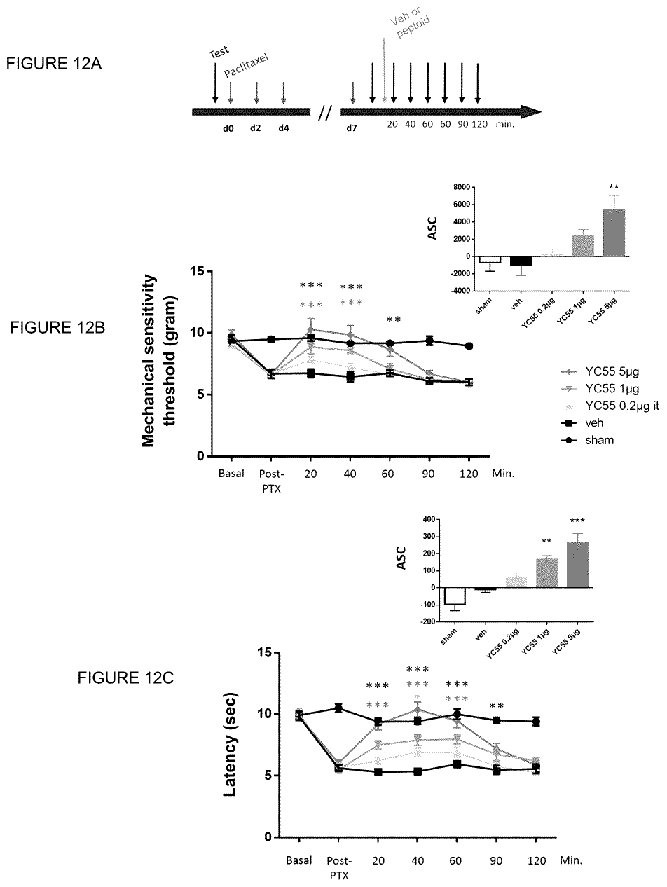

YC55 also demonstrated a dose-dependent analgesic effect in a rat model of paclitaxel-induced neuropathy when administered i.t. (FIG. 12).

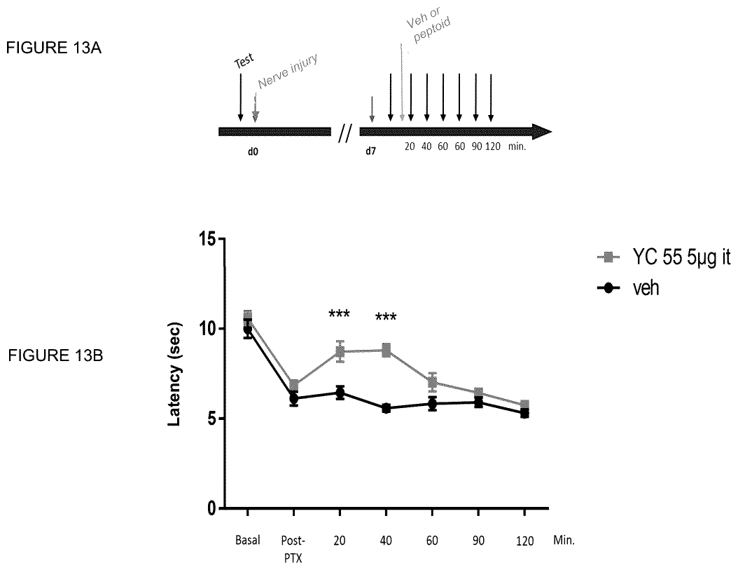

Intrathecal administration of YC55 showed an antihyperalgesic effect in a model of neuropathy induced by traumatic sciatic nerve injury (FIG. 13), suggesting that the strategy of disrupting the TRIP8b interaction may have an analgesic effect in several neuropathic pain states.

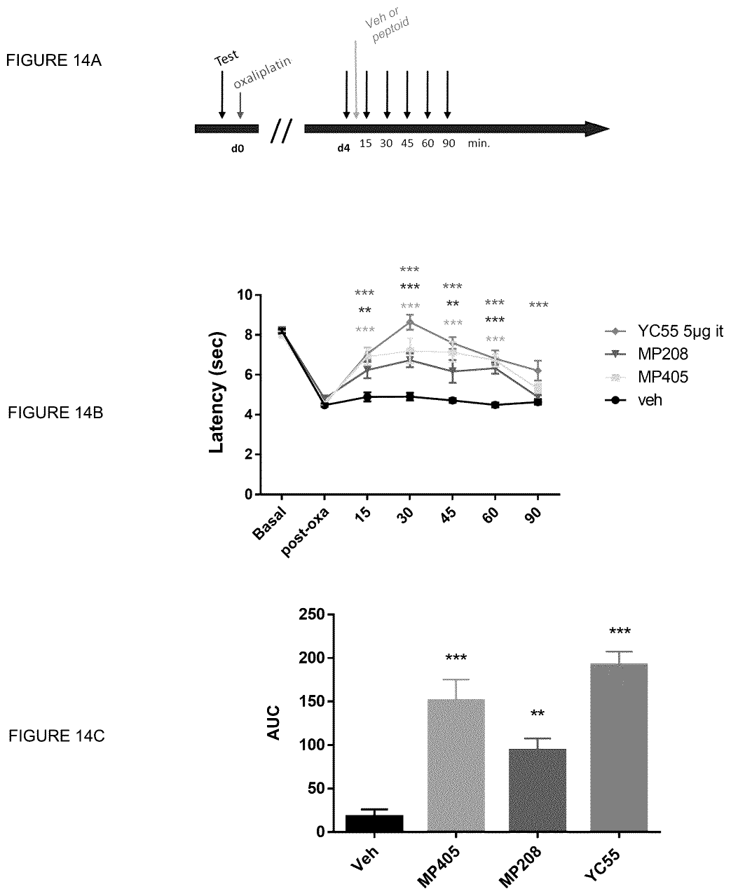

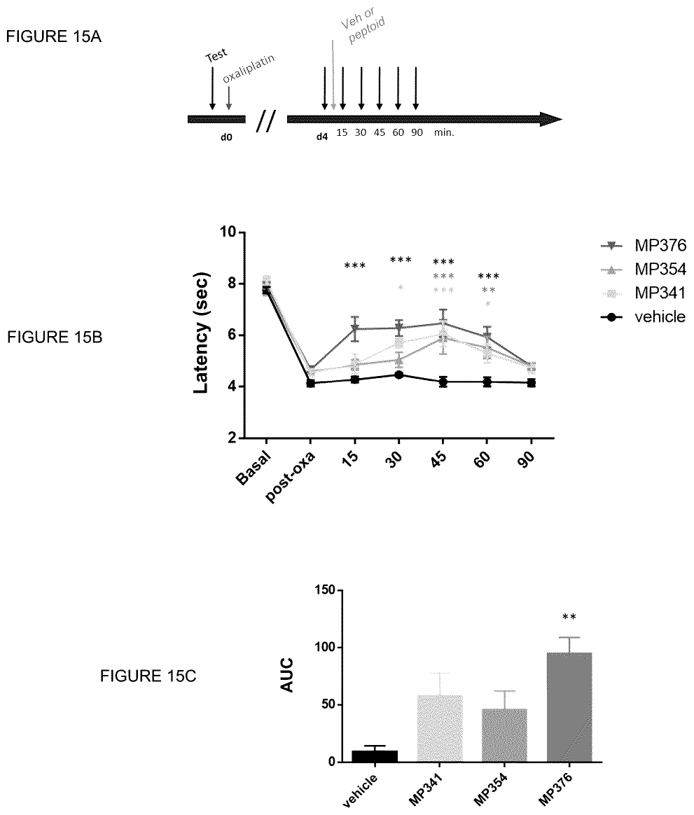

Compounds MP208, MP405 (5 μg, i.t.) and MP341, MP354, MP376 (10 mg/kg, s.c.) demonstrated an analgesic effect in an oxaliplatin-induced neuropathy model in mice (FIG. 14 and FIG. 15 respectively).

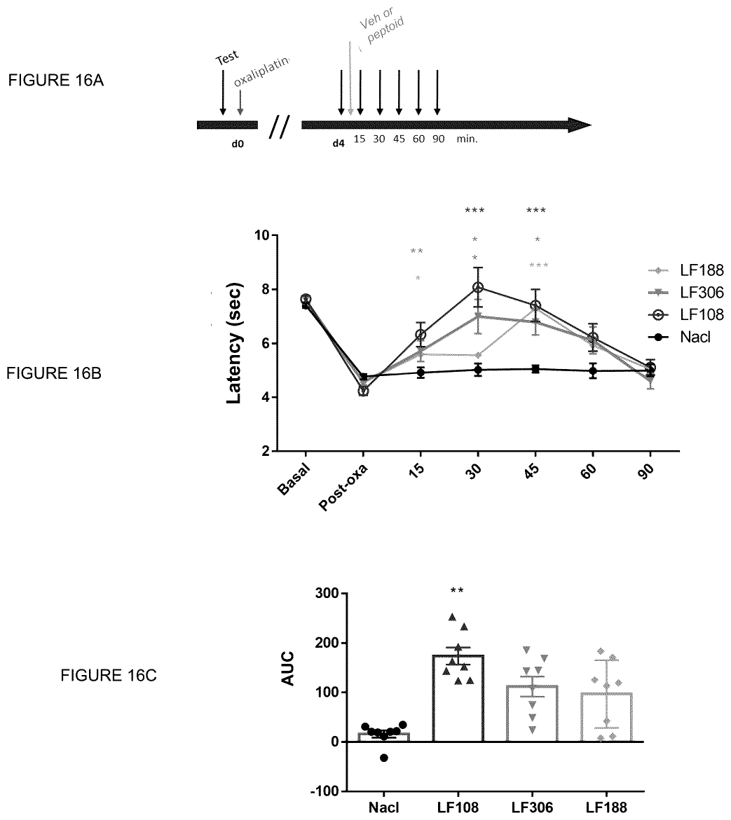

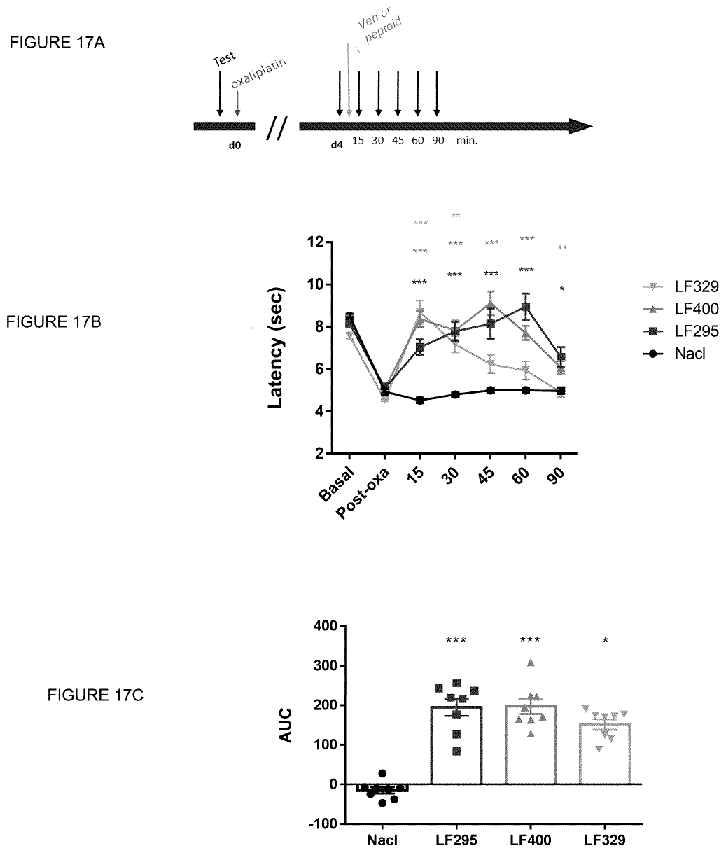

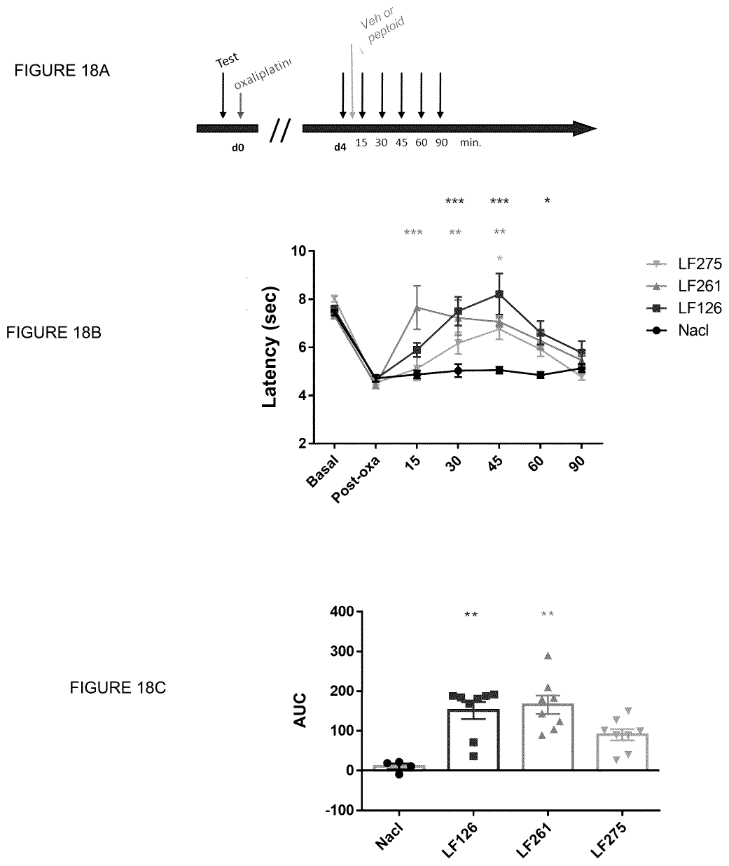

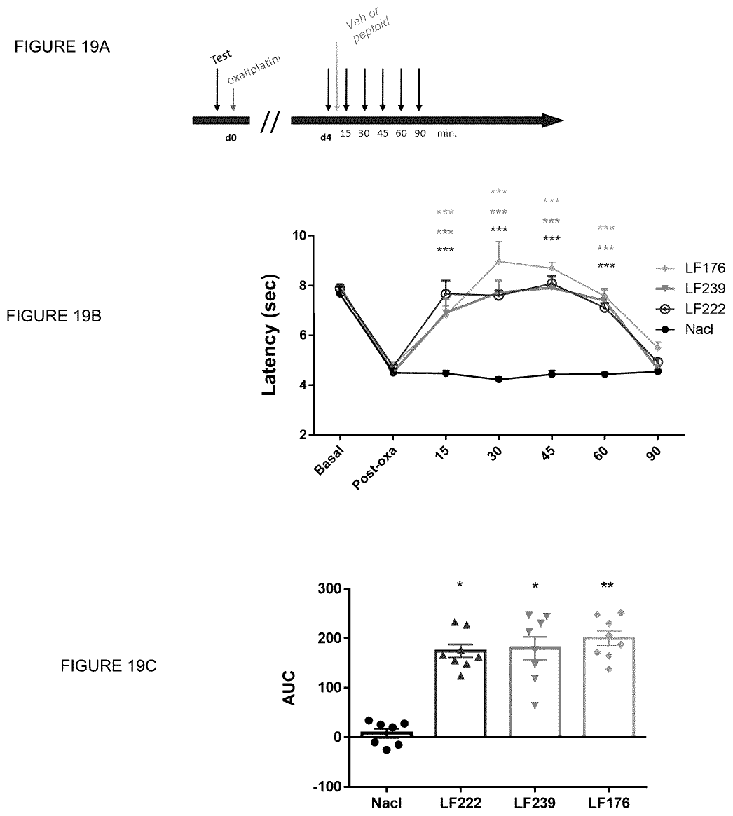

Similarly, compounds LF108, LF306, LF188 (FIG. 16), LF400, LF329, and LF295 (FIG. 17), LF275, LF261, and LF126 (FIG. 18), LF239, LF222, and LF176 (FIG. 19), demonstrated an analgesic effect (5 μg, i.t.) in an oxaliplatin-induced acute neuropathy model in mice.

### B—Trimeric Peptomimetics

The trimeric peptomeric compounds are also mimics of the carboxy-terminal sequence of the HCN2 channel. They were designed based on the extensive X-ray structure study of a co-crystal of TRIP8b with the C-terminal region of HCN2 (bp: 4EQF).

These ligands have a formula which is represented by the formula LF369 below or the following general formula (II):

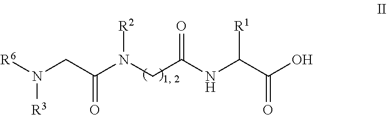

where
R¹, R² and R³ are as previously defined;
R⁶ is H, R³, CO R⁵ (where R⁵ is as defined above), or R⁷;
R⁷ is independently one of:

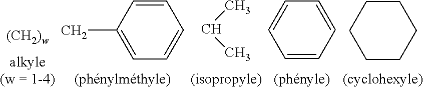

The compounds are peptide-peptoid hybrid oligomers (peptomers) (Ostergaard et al., 1997) [17]. They comprise three units including an α-amino acid positioned at the C-terminus of the sequence, followed by two peptoid residues.

The syntheses were performed in solution from an α-amino acid protected as a tert-butyl ester (Xaa-tBu, the L-Leu-tBu in the case of the synthesis of LF369). These syntheses can also be performed on resin as previously described for tetrameric peptidomimetics. From this unit, the elongation was performed as for the molecules corresponding to the general formula (I), by “submonomer chemistry” approaches. When the core unit is of the β-peptoid type, the two steps necessary for its construction are the reaction of Xaa-tBu with acryloyl chloride, followed by an aza-Michael reaction to introduce the desired side chain (R²) to the backbone. The third α-peptoid unit is constructed by acylation of the dimer with bromoacetic acid bromide followed by substitution of the bromine atom with an amine. In the case where R⁶ is H, the amine used is a primary amine R³NH₂. When R⁶ is COR⁵, the secondary amine obtained after treatment with R³NH₂ is acylated with an acid chloride, acid anhydride or carboxylic acid under coupling conditions. Compounds in which R⁶=R⁷ are obtained by treating the N-terminally bromoacetylated dimers with a secondary amine R³R⁷NH. They can also be obtained by reductive amination of compounds with NHR³ at the N-terminus. The final step is to deprotect the side chains and the C-terminus by treatment with TFA.

### The Precise Experimental Protocol for the Synthesis of LF369 is as Follows:

In a flask, L-leu-tBu (1.74 g, 7.8 mmol) is dissolved in 45 mL of anhydrous THF. The flask under an inert argon atmosphere is cooled to 0° C. Triethylamine (2.3 mL, 31.4 mmol, 4 equiv) is added, followed by acryloyl chloride (0.76 mL, 9.4 mmol, 1.2 equiv). After 3 h stirring at 0° C. a thin layer chromatography (50/50 cyclohexane/AcOEt) indicates full conversion. The salts formed are removed by filtration, the filtrate is evaporated under vacuum and the product obtained is purified by silica gel chromatography (50/50 cyclohexane/AcOEt). The acrylamide is isolated in 93% yield (1.76 g, 7.27 mmol) as a white solid.

Aza-Michael reaction. The acrylamide formed previously (1.76 g, 7.27 mmol) is dissolved in 20 mL of ethanol. Ethanolamine with the alcohol function protected as tertbutyldimethylsilyl ether (2.68 g, 15.28 mmol, 2.1 equiv.) is added and the reaction mixture is heated to reflux for 60 h. The solvent is then evaporated and the reaction crude purified by silica gel chromatography (90/10/0.1 AcOEt/MeOH/Et3N) affording the dimer in 90% yield (2.74 g, 6.58 mmol) as a yellowish oil.

Acylation reaction of the dimer. The dimer (2.74 g, 6.58 mmol) is dissolved in 10 mL THF, the reactor is inerted with argon and then cooled to −20° C. Triethylamine (1.1 mL, 7.89 mmol, 1.2 equiv.) is added to the solution, followed by the addition of 2-bromoacetyl bromide (0.62 mL, 7.89 mmol, 1.2 equiv.). After 3 h of stirring at −20° C., the reaction mixture is filtered. The resulting filtrate is evaporated and the reaction crude is chromatographed on silica gel (60/40/0.1 Cyclohexane/AcOEt/Et₃N). The bromoacetylated dimer is isolated in 67% yield (2.36 g, 4.4 mmol) as a yellowish oil.

Substitution reaction. The previously obtained product (2.36 g, 4.4 mmol) is dissolved in 10 mL THF. Triethylamine (1.2 mL, 8.8 mmol, 2 equiv.) and then protected ethanolamine H₂NCH₂CH₂OTBDMS (2.31 g, 13.2 mmol, 3 equiv.) are added to the solution. The reaction medium under argon atmosphere is stirred overnight at room temperature. The reaction mixture is then filtered and the filtrate obtained is evaporated. The crude product is purified by silica gel chromatography (95/5/0.1 AcOEt/MeOH/Et₃N) leading to the trimer in 83% yield (2.30 g, 3.64 mmol) as an oil.

Acetylation of the trimer. The resulting amine (2.3 g, 3.64 mmol) is dissolved in 20 mL of ethyl acetate. Triethylamine (2.02 mL, 14.5 mmol, 4 equiv.) and then acetic anhydride (2.75 mL, 29 mmol, 8 equiv.) are added to the solution. The reaction is placed under argon at room temperature for 60 h. After evaporation of the solvent the crude obtained is chromatographed on silica gel (90/10/0.1 AcOEt/cyclohexane/Et3N) leading to the N-acetylated trimer in 92% yield (2.27 g, 3.36 mmol) as a colorless oil.

Deprotection of the side chains and C-terminus. The trimeric compound (502 mg, 0.776 mmol, 1 equiv.) is dissolved in 4 mL of a TFA/CH2Cl2/H2O (47.5:47.5:5) mixture. After 2 h of stirring at room temperature, 10 mL of dichloromethane is added. The reaction medium is evaporated under vacuum and then 5 co-evaporations with dichloromethane are performed. The product is then redissolved in 5 mL of distilled water and left under stirring at room temperature for 2 h. The solvent is evaporated and then five co-evaporations with toluene are performed leading to LF369 (362 mg) as an oil.

### Side Chains

R¹, R² and R³ are as previously defined;
R⁶ is R³, H, COR⁵ (where R⁵ is as previously defined), or R⁷;
R⁷ is independently one of:

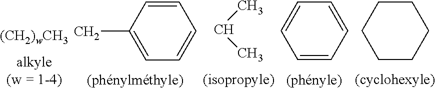

Representative Synthetic Scheme for the Solution Synthesis of Peptoids of General Formula (II), e.g. LF369

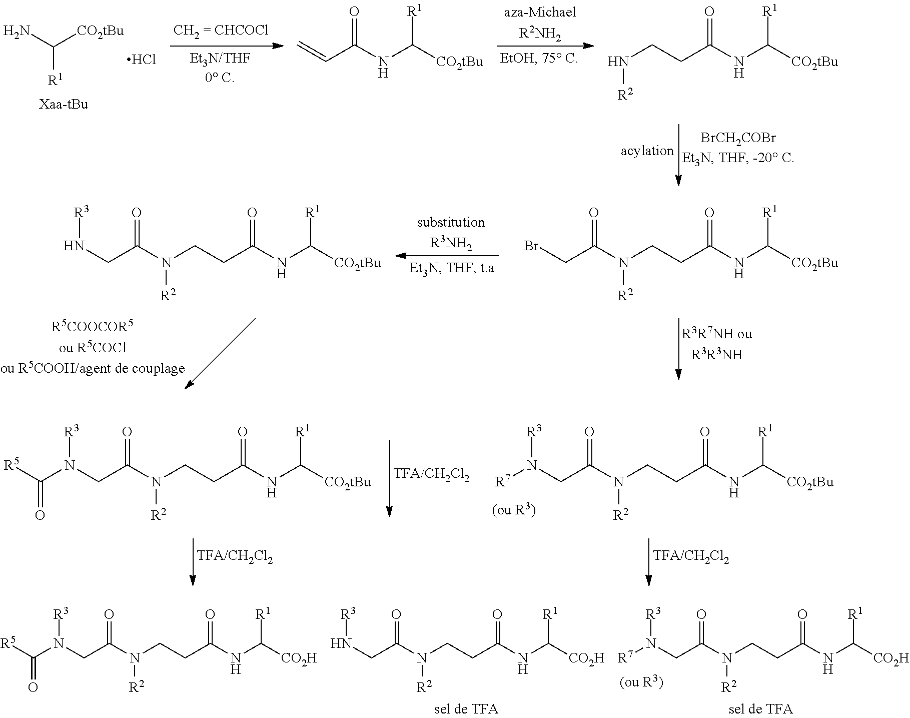

Representative Synthetic Scheme for the Supported Synthesis of Peptoids of General Formula (II), e.g. LF369

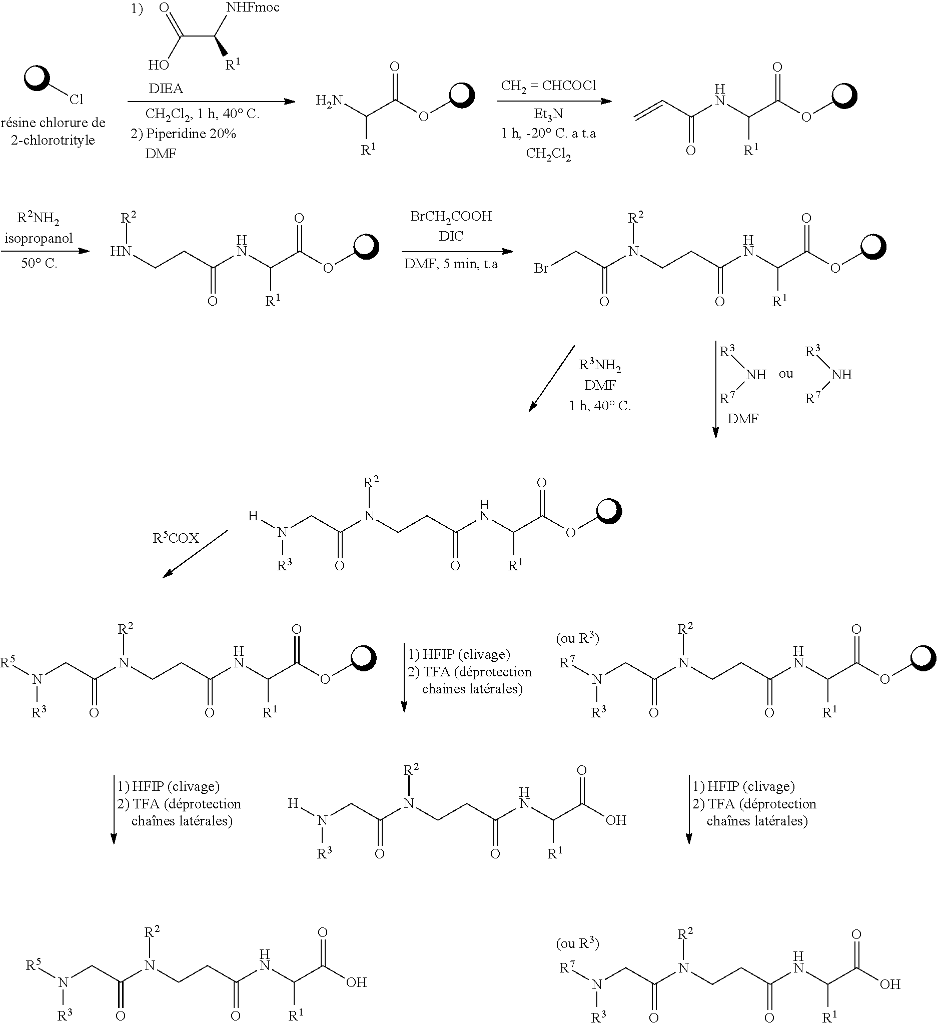

### Synthesized Molecule

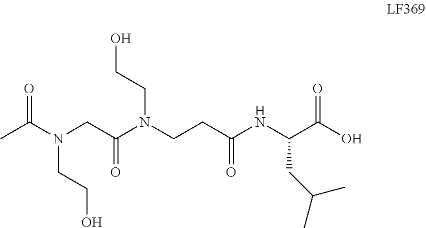 [SMILES: CC(=O)N(CCO)CC(=O)N(CCO)CCC(=O)N[C@@H](CC(C)C)C(=O)O]

### Synthesis

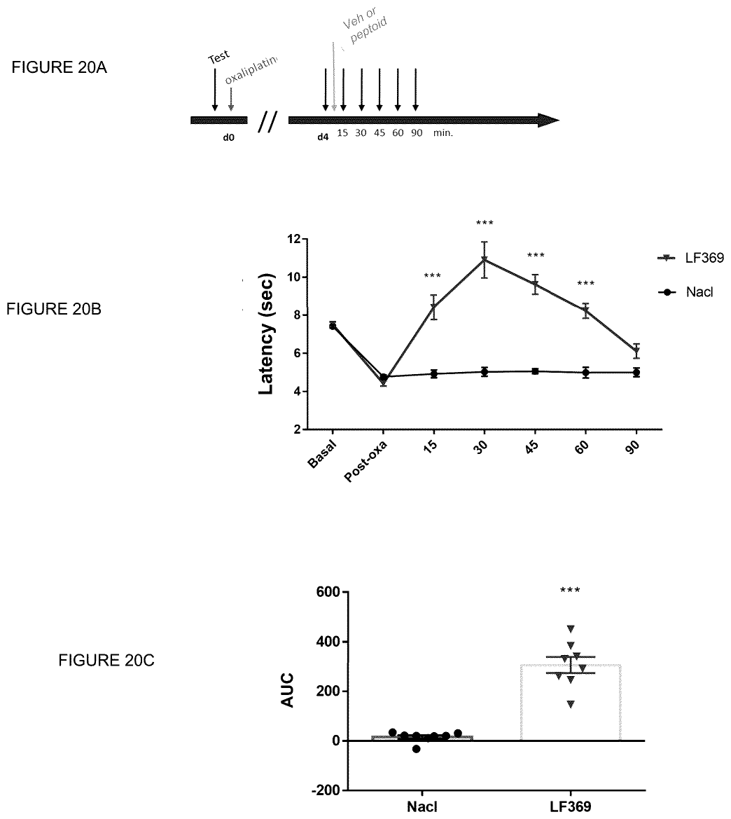

The compound LF369 (5 μg, i.t.) demonstrated an analgesic effect in a mouse model of oxaliplatin-induced neuropathy (FIG. 20).

### LIST OF REFERENCES

1. Finnerup et al., Lancet Neurol., 14(2): 162-173, 2015
2. von Hehn et al., Neuron, 73(4): 638-652, 2012
3. Hershman et al., J. Clin. Oncol., 32(18): 1941-1967, 2014
4. Ludwig et al., Nature, 393(6685): 587-591, 1998
5. Santoro et al., Cell, 93(5): 717-729, 1998
6. Seifert et al., Proc. Natl. Acad. Sci. USA, 96(16): 9391-9396, 1999
7. Dunlop et al., Curr. Pharm. Des., 15(15): 1767-1772, 2009
8. Lewis et al., Mol. Cell Neurosci., 46(2): 357-367, 2011
9. Descoeur et al., EMBO Mol. Med., 3(5): 266-278, 2011
10. Young et al., Pain, 155(9): 1708-1719, 2014
11. Lewis et al., J. Neurosci., 29(19): 6250-6265, 2009
12. Bankston et al., Proc. Natl. Acad. Sci. USA, 109: 7899, 2012
13. Han et al., J. Biomol. Screen, 20(9): 1-8, 2015
14. Janssen et al., Arzneimittelforschung, 13: 502-507, 1963
15. Randall et Selitto, Arch. Int. Pharmacodyn. Ther., 111(4): 409-419, 1957
16. Kim et Chung, Pain, 50(3): 355-363, 1992
17. Ostergaard et al., Molecular Diversity, 3: 17-27, 1997(
18. Simon et al., Proc. Natl. Acad. Sci USA, 89: 9367-9371, 1992
19. Hamper et al., J. Org. Chem., 63: 708-718, 1998
20. Zuckermann et al., J. Am. Chem. Soc., 114: 10646-10647, 1992
21. Culf et al., Molecules, 15: 5282-5335, 2010
22. Miller et al., Drug Dev. Res., 35: 20-32, 1995
23. Kwon et al., J. Am. Chem. Soc., 129: 1508-1509, 2007
24. Vollrath et al., Organic & Biomolecular Chemistry, 11: 8497-8201, 2013
25. Li et al., Cellular & Molecular Immunol., 7: 133-142, 2010
26. Chongsiriwatana et al., Proc. Natl. Acad Sci. USA, 105: 2794-2799, 2008
27. Mesirca et al., Nat. Commun., 5: 4664.doi:10.1038/ncomms5664, 2014
28. Marger et al., Channels (Austin), 5(3): 241-250, 2011

1. Peptoid of the following general formula (I): 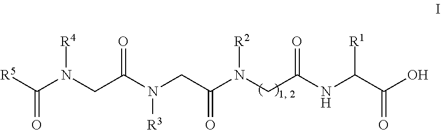
where
R¹ is independently CH₃ (L-Ala), CH(CH₃)₂ (L-Val), CH(CH₃)CH₂CH3 (L-Ile), CH₂CH(CH₃)₂ (L-Leu and D-Leu), CH₂C(CH₃)₃, C(CH₃)₃, CH₂(cyclobutyl);
R² and R³ are independently selected from: CH₂CH(CH₃)₂, CH₂CH₂NH₂, CH₂CH₂OH, CH₂CH₂CH₂OH, CH₂CH(OH)CH₃, CH₂CONH₂, CH₂CH₂CONH₂;
R⁴ is an aliphatic or aromatic group selected from: 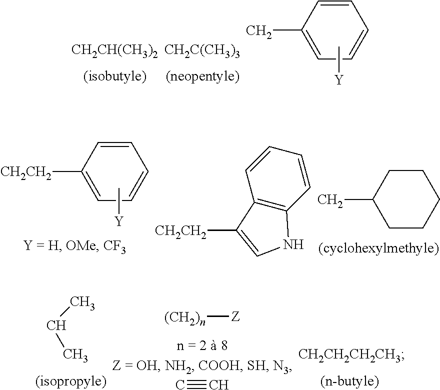
R⁵ is independently CH₃, CH(CH₃)₂, CH₂C₆H₅, C₆H₄X (where X in the ortho, meta or para position is independently H, OMe, CF₃, CH₃, CH₂CH₃, F, Br, Cl, NO₂), 3-indolyl, 3-quinolinyl.

2. Peptoid according to claim 1 of the following formula:

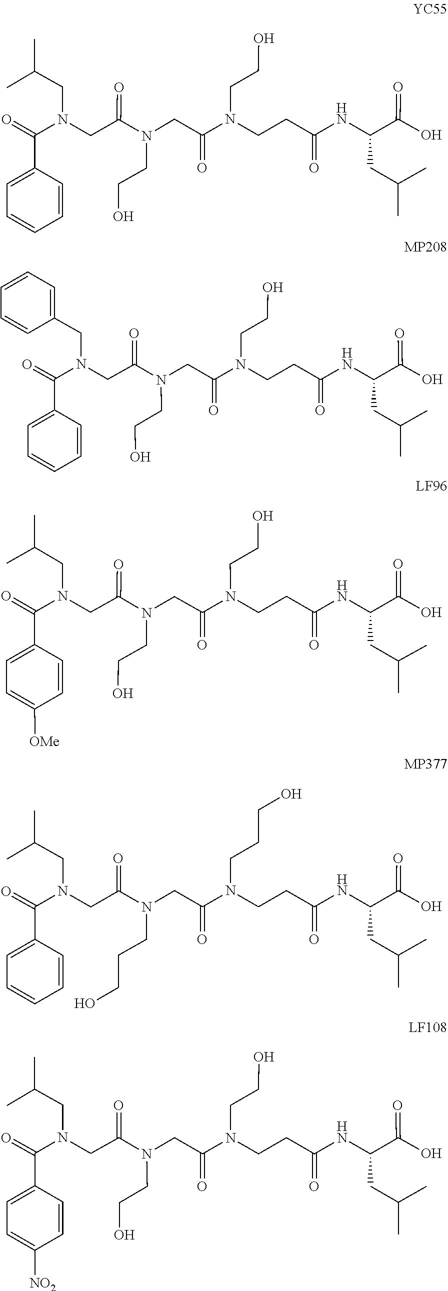

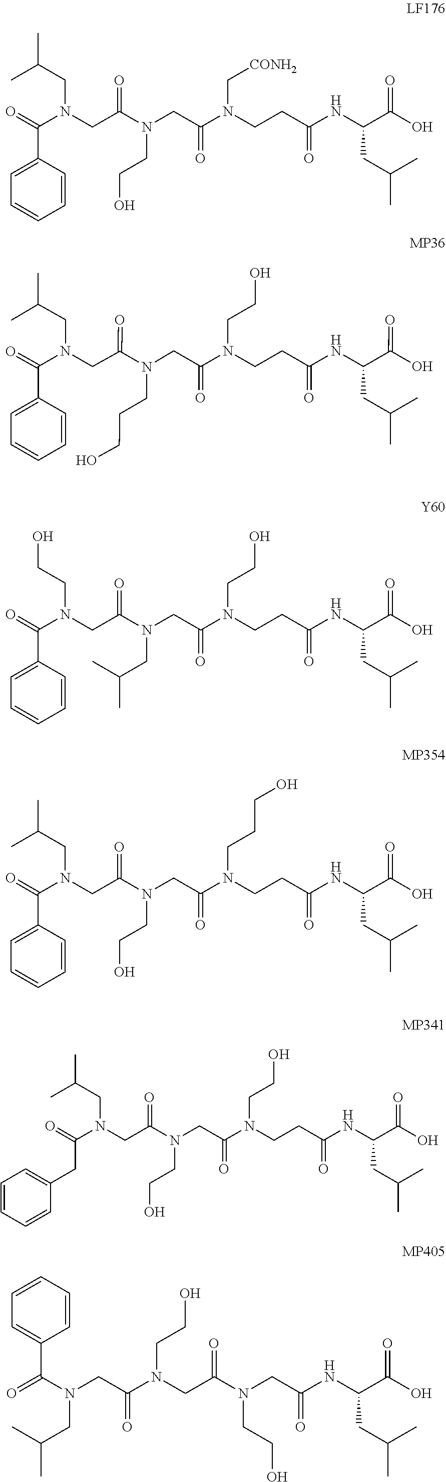

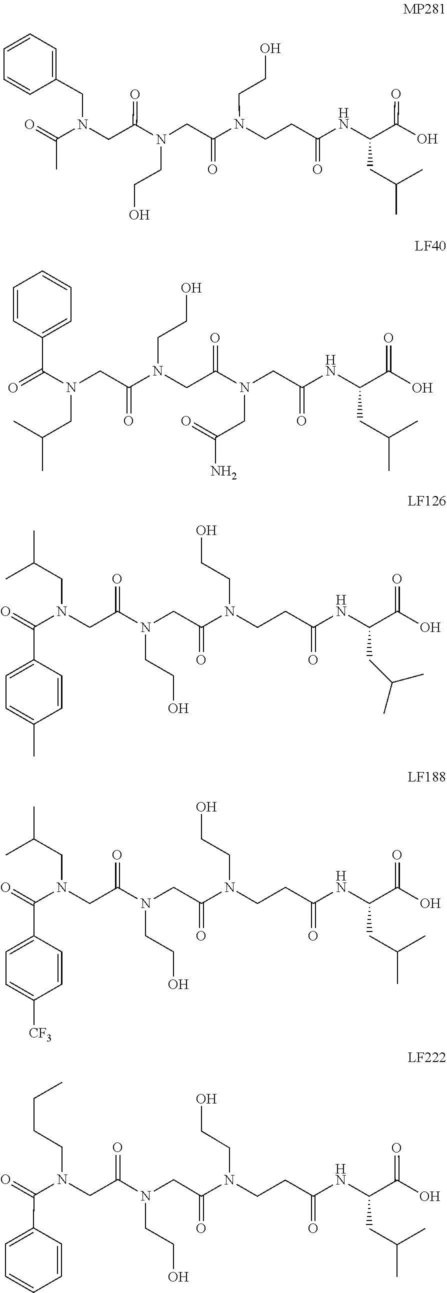

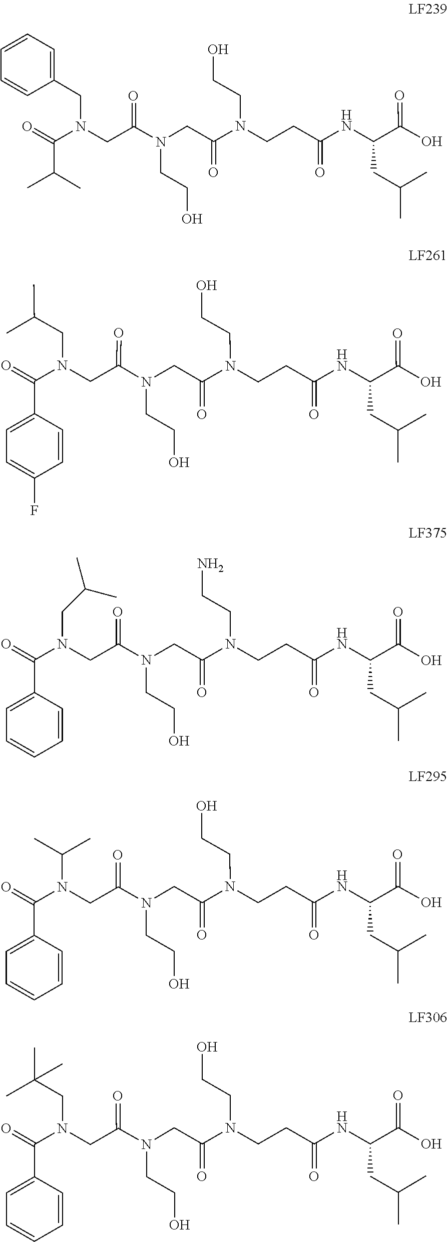 [SMILES: CC(C)C[C@H](NC(=O)CCN(CCO)C(=O)CN(CCO)C(=O)CN(Cc1ccccc1)C(=O)C(C)C)C(=O)O ; CC(C)C[C@H](NC(=O)CCN(CCO)C(=O)CN(CCO)C(=O)CN(CC(C)C)C(=O)c1ccc(F)cc1)C(=O)O ; CC(C)C[C@H](NC(=O)CCN(CCN)C(=O)CN(CCO)C(=O)CN(CC(C)C)C(=O)c1ccccc1)C(=O)O ; CC(C)C[C@H](NC(=O)CCN(CCO)C(=O)CN(CCO)C(=O)CN(C(=O)c1ccccc1)C(C)C)C(=O)O ; CC(C)C[C@H](NC(=O)CCN(CCO)C(=O)CN(CCO)C(=O)CN(CC(C)(C)C)C(=O)c1ccccc1)C(=O)O]

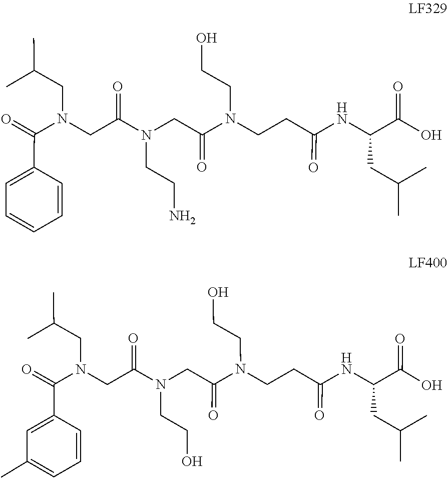

3. Peptoid of the following general formula (II): 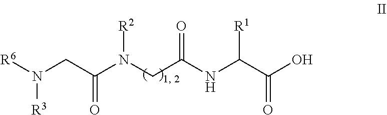
wherein
R¹, R² and R³ are as defined in claim 1;
R⁶ is R³, H, COR⁵ (wherein R⁵ is as defined above) or R⁷;
R⁷ is independently one of:

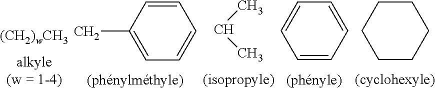

4. A pharmaceutical composition comprising a peptoid of claim 1, and a pharmaceutically acceptable carrier.

5. A method comprising:
administering the peptoid of claim 1 to a subject in need thereof as a medicament.

6. A method comprising:
administering the peptide of claim 1 to a subject in need thereof to prevent or treat chronic pain.

7. The method according to claim 6, wherein the chronic pain is of neuropathic origin.

8. A method comprising:
administering the pharmaceutical composition of claim 4 to a subject in need thereof as a medicament.

9. A method comprising:
administering the pharmaceutical composition of claim 4 to a subject in need thereof to prevent or treat chronic pain.

10. The method according to claim 9, wherein the chronic pain is of neuropathic origin.
